(19) United States
(12) Patent Application Publication
(10) Pub. No.: US 2014/0163557 A1
(43) Pub. Date: Jun. 12, 2014

(54) GUIDING SYSTEM

(71) Applicant: CARBOFIX ORTHOPEDICS LTD., Herzlia Pituach (IL)

(72) Inventors: Mordechay Beyar, Caesarea (IL); Oren Man, Kfar-Shemaryahu (IL); Sasi Solomon, Herzlia (IL)

(21) Appl. No.: 14/117,642

(22) PCT Filed: May 15, 2012
(86) PCT No.: PCT/IB12/52437

§ 371 Date: Feb. 19, 2014

Related U.S. Application Data
(60) Provisional application No. 61/486,283, filed on May 15, 2011.
(60) Provisional application No. 61/514,500, filed on Aug. 3, 2011.

(51) Int. Cl. A61B 17/17; A61B 17/16

(52) U.S. Cl. CPC A61B 17/17; A61B 17/1637
USPC 606/80; 606/96

(57) ABSTRACT

A method and system for guiding a drill or nail to intersect both bone and an aperture in an implanted implant. In an exemplary embodiment of the invention, a vector is defined interconnecting a path of a tool or cross-implant and the bone and implanted implant. In an exemplary embodiment of the invention, the vector is defined using a light source and two sensors or a light source and a sensor which is on an opposite side of said bone from said source.

### RELATED APPLICATION/S

This application claims the benefit of priority and under 35 USC 119 of U.S. Provisional Patent Application No. 61/486,283 to Beyar, filed May 15, 2011 and U.S. provisional application 61/514,500 filed Aug. 3, 2011.

The contents of all of the above documents are incorporated by reference as if fully set forth herein.

### FIELD AND BACKGROUND OF THE INVENTION

The present invention, in some embodiments thereof, relates to a system and method for aiming tools and/or implants at a target location in a bone and, more particularly, but not exclusively, to a system for guiding a bone drill to an aperture in an implant and to a bone.

Intramedullary fixation provides an alternative to open reduction and fixation of a variety of fractures. The objective of this closed technique as compared to open techniques is to provide fixation with minimal trauma, reduced risk of infection, and reduced blood loss.

In general, the intramedullary nails are rod-shaped, rigid, devices, and may be secured (interlocked) to the bone using one or more locking element, such as transverse screws at one or both nail ends. Such locking elements are placed through holes located along the nail, usually at its proximal and/or distal end.

One of the issues associated with intramedullary nailing of long bone fractures, relates to the insertion of the locking elements. In order to implant such a locking element (e.g., a screw), so that it will provide for connection of the nail to the bone, a hole has to be drilled through the bone, in line with the location of the desired hole in the nail. While certain aiming devices that connect to the nail insertion handle are available for the alignment of the drill with the proximal interlocking holes of the nail, it is more difficult to provide such aiming devices for alignment of the drill with the distal interlocking holes of the nail. Proper alignment of the drill with the hole within the nail is desired in order to avoid nail shaving by the drill. In addition, if not properly aligned, additional drill hole(s) will be required, thus reducing bone strength. Misalignment of the drill with the nail hole may also result in mal-placement of the screw and in damage to the surrounding tissue.

Many of the methods that are used for distal screws placement increase operative time, as the location thereof is generally time consuming. Also, such methods often involve an increased exposure to radiation, such as X-rays, which is used to assist in the proper location of the holes in the nail. The combination of prolonged operation time and X-ray exposure poses a health concern to both the surgeon and the patient, as well as to any assisting personnel in the operating theater.

Optical, light-emitting, devices for targeting distal holes of intramedullary nails have been suggested in the past.

U.S. Pat. No. 5,540,691 describes an apparatus and a method for detecting the location of the transverse holes of an intramedullary nail inserted into a long bone, and for alignment of a drill to the holes. This patent describes a device with a light source at its distal end which emits in the visible or infrared (IR) spectrum. This device is inserted into the nail such that the light source is placed adjacent to the nail transverse holes. The emitted radiation is visually detected by direct vision or with the help of a camera and a monitor. The surgeon aligns the drill with the emitted radiation observed.

WO 2007/131231 A2 describes an intramedullary transillumination apparatus and a surgical kit and a method for accurate placement of locking screws in intramedullary rodding of long bones. The light emitted from the light source, which is inserted into the intramedullary nail, is detected by direct eye vision, or using an arthroscope with or without an external camera and monitor.

WO 2009/131999 A2 describes a light delivery structure for use in intramedullary transillumination apparatus and a method for its producing. The light delivery structure is placed within the intramedullary rod.

U.S. Pat. Nos. 6,081,741 and 6,895,266 describe a device and a method for surgical site location using a light emitter and an array of light sensors with a display. The light emitter is placed within the target organ and is detected by the sensors. The signal is then processed to provide indication of the relative direction of the sensors as compared to the emitter. The sensors array may be connected to a drill guide to assist in orthopedic surgery.

### SUMMARY OF THE INVENTION

The present invention in some embodiments thereof relates to orienting a directional tool with a bone and with an aperture in an orthopedic implant. In an exemplary embodiment of the invention, the alignment is by defining a vector through the bone and the aperture using a light source and one or more detectors.

There is provided in accordance with an exemplary embodiment of the invention, a guiding system adapted to guide drilling through an aperture in an orthopedic implant, comprising:

a frame rigidly interconnecting at least two optical elements; and

a radiation source and a radiation detector, at least one of which is one of said at least two optical elements;

circuitry which powers said radiation source to generate a radiation beam which is detected by said radiation detector after passing through human bone and overlying soft tissue, wherein said circuitry generates an indication of a change in intensity due to alignment of said beam with said aperture and the optical elements,

such that said circuitry generates a signal indicating if a vector pathway for light interconnects said two optical elements through said aperture. Optionally, said circuitry generates an indication of a correction to apply to improve said alignment. Optionally or alternatively, the system comprises a drill guide rigidly coupled to said frame. Optionally, the system comprises said drill guide comprises at least one of said optical elements integrated therein, to receive or transmit said radiation beam at a distal end thereof.

In an exemplary embodiment of the invention, said drill guide is configured to move along a drilling axis thereof relative to said frame to a different rigidly interconnected position thereon.

In an exemplary embodiment of the invention, said drill guide is configured to move along a direction other than along a drilling axis thereof relative to said frame to a different rigidly interconnected position thereon.

In an exemplary embodiment of the invention, at least one of said optical elements is configured to move relative to said frame to a different rigidly interconnected position thereon.

In an exemplary embodiment of the invention, said optical elements are configured to be on opposite sides of said aperture.

In an exemplary embodiment of the invention, said optical elements are configured to be on a same side of said aperture.

In an exemplary embodiment of the invention, said optical elements are both radiation detectors.

In an exemplary embodiment of the invention, at least one of said optical elements is a radiation source.

In an exemplary embodiment of the invention, said radiation source is configured to pass within a channel or groove in said orthopedic implant.

In an exemplary embodiment of the invention, said optical element operates in visible or IR or NIR wavelengths.

In an exemplary embodiment of the invention, said optical element operates in visible and/or UV wavebands.

In an exemplary embodiment of the invention, said optical element operates in ionizing radiation wavebands.

In an exemplary embodiment of the invention, said system is configured to operate with an intramedullary nail for a leg bone and through at least 3 cm of soft tissue.

In an exemplary embodiment of the invention, said system is configured to operate with a bone plate and through at least 1 cm of soft tissue.

In an exemplary embodiment of the invention, said detector comprises an array of detectors.

In an exemplary embodiment of the invention, said detector comprises at least two spaced apart detectors with an aperture therebetween.

In an exemplary embodiment of the invention, said circuitry is configured to detect a difference between light passing through at least 0.5 cm of soft tissue and a layer of cortical bone and light passing through a similar thickness of soft tissue and bone, but being blocked by an orthopedic implant.

In an exemplary embodiment of the invention, the system is adapted to be mounted on a drill and moved free-hand therewith.

There is provided in accordance with an exemplary embodiment of the invention, a method of detecting a location of an aperture in an orthopedic implant, comprising:

using two rigidity coupled optical elements, passing a radiation beam from one optical element through the aperture and to the other element or from said implant to both said elements; and

generating an indication of aperture location or detection based on said detection of said beam.

In an exemplary embodiment of the invention, the method comprises generating said beam inside said implant.

In an exemplary embodiment of the invention, the method comprises passing said beam from one side of the body, through soft tissue and bone and the aperture, to another side of said body.

In an exemplary embodiment of the invention, generating an indication comprises moving said beam relative to said aperture along an axis of said implant and/or transverse to said axis and/or providing relative rotation between said beam and said implant.

In an exemplary embodiment of the invention, generating an indication comprises acquiring a plurality of detections of radiation beams at different relative position sand/or orientations to said aperture.

In an exemplary embodiment of the invention, the method comprises advancing a drill guide along said beam using said indication. Optionally, the method comprises drilling through said drill guide using a bone drill.

There is provided in accordance with an exemplary embodiment of the invention, a bone drilling guiding system adapted to guide drilling through an aperture in an orthopedic implant, comprising:

a frame adapted to rigidly couple to a human body and to rigidly interconnecting at least one optical element and a drill guide adapted to be inserted through soft tissue; and

a radiation source and a radiation detector, at least one of which is one of said at least one optical element;

circuitry which powers said radiation source to generate an electromagnetic radiation beam which is detected by said radiation detector after passing through human bone and overlying soft tissue, wherein said circuitry generates an indication of a change in intensity due to alignment of said beam with said aperture. Optionally, said drill guide comprises at least one of said optical elements integrated therein, to receive or transmit said radiation beam at a distal end thereof.

There is provided in accordance with an exemplary embodiment of the invention, a method of drilling a hole in a bone to match an aperture in an orthopedic implant, comprising:

detecting a radiation beam using a frame rigidly coupled to a drill guide;

advancing said drill guide into soft tissue responsive to said detection; and

drilling via said drill guide.

There is provided in accordance with an exemplary embodiment of the invention, a method of identifying a location of an aperture in an orthopedic implant, comprising:

generating a radiation beam outside the body and aiming it at said implant;

detecting said beam at a plurality of axial and/or transaxial locations relative to said aperture; and

identifying said aperture from said multiple detectings. Optionally, said detecting comprises detecting using an array of sensors. Optionally or alternatively, said detecting comprises moving at least one detector relative to said aperture.

There is provided in accordance with an exemplary embodiment of the invention, a system for locating of an aperture in an orthopedic implant, comprising:

a light source which generates a radiation beam outside the body and aims it at said implant;

at least one detector which detects said beam; and

circuitry configured to combine multiple detection results and produce a location indication for said aperture from said results.

There is provided in accordance with an exemplary embodiment of the invention, a method of identifying a location of an aperture in an orthopedic implant, comprising:

generating a radiation beam outside the body and aiming it at said implant;

detecting said beam inside said implant or at an opposite side of said body using a detector; and

identifying said aperture location based on a vector connecting a location of said beam generation and a location of said beam detection.

There is provided in accordance with an exemplary embodiment of the invention, a method of anatomical imaging, comprising:

transmitting non-ionizing electromagnetic radiation through soft tissue to bone at an anatomical location;

receiving said radiation after reflection from or transmission through said bone; repeating said transmitting for a plurality of anatomical locations to collect transmission data; and

reconstructing an image of said bone from said data. Optionally, said transmitting comprises transmitting at a plurality of angles relative to said anatomical location. Optionally or alternatively, said reconstructing comprises reconstructing a location of an aperture in an orthopedic implant of said bone.

In an exemplary embodiment of the invention, the method comprises identifying an abnormality based on amplitude of a detection at an angle relative to a beam of said radiation, compared to an expected amplitude at said angle.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a light source, in accordance with some embodiments of the present invention, placed within the cannulation of an intramedullary nail;

FIGS. 2A-2C are schematic illustrations of various designs of a light source, in accordance with some embodiments of the present invention;

FIG. 3 is a schematic illustration of a light source placed within the cannulation of an intramedullary nail, in accordance with some embodiments of the present invention;

FIG. 4A is a schematic illustration of a targeting system including a sensors-assembly and a tool guide, in accordance with some embodiments of the present invention;

FIG. 4B shows the targeting system of FIG. 4A mounted on a limb of a patient, in accordance with an exemplary embodiment of the invention;

FIG. 4C is a flowchart of a method of implanting a distal locking element, in accordance with an exemplary embodiment of the invention;

FIG. 5 is a schematic illustration of an exemplary arrangement of sensors in a sensors-assembly such as shown in FIG. 4A, in accordance with some embodiments of the present invention;

FIG. 6 is a cross-sectional view of the targeting system of FIG. 4A, after advance of the tool guide thereof to a bone, in accordance with some embodiments of the present invention;

FIG. 7 shows a design for a targeting system with an external light source and a sensor on an opposite side of a bone therefrom, in accordance with an exemplary embodiment of the invention;

FIG. 8 shows a design for a targeting system with an external light source and a sensor on a same side of a bone, and with a tool guide on an opposite side of a bone therefrom, in accordance with an exemplary embodiment of the invention;

FIG. 9 shows a design for a targeting system with an external light source and a sensor and a tool guide all on a same side of a bone, in accordance with an exemplary embodiment of the invention;

FIG. 10 shows a design for a targeting system with an array sensor configuration and which is optionally moved out of the way to make room for a tool guide, in accordance with an exemplary embodiment of the invention;

FIGS. 11A-11C shows a design for a targeting system with an array sensor configuration in various states of operation and relative location of sensor array, tool guide and bone, in accordance with an exemplary embodiment of the invention; and

FIGS. 12A and 12B illustrate a targeting system mounted on a power drill, in accordance with some embodiments of the present invention.

### DESCRIPTION OF EXEMPLARY EMBODIMENTS OF THE INVENTION

Overview

The present invention in some embodiments thereof relates to orienting a directional tool with an aperture in an orthopedic implant in the bone. In an exemplary embodiment of the invention, the alignment is by defining a vector through the bone and the aperture in the orthopedic implant and optionally overlying soft tissue, using a radiation source and one or more detectors.

An aspect of some embodiments of the invention relates to orienting a tool with an aperture in an orthopedic implant. In an exemplary embodiment of the invention, the tool guide is aligned (e.g., lateral position and/or orientation), by aligning it with a vector of a desired tool pathway. In an exemplary embodiment of the invention, the tool pathway includes a position of an aperture to be formed in a bone. Optionally, the pathway includes two apertures to be formed in the bone

In an exemplary embodiment of the invention, the alignment includes translation along a bone axis and/or perpendicular to a bone axis (e.g., for elongate bones) and/or one or two dimensional offset (e.g., a projection thereof) in a plane perpendicular to the tool pathway.

In an exemplary embodiment of the invention, the alignment includes an orientation of an axis of said tool guide with an axis of the bone, for example, to define a desired insertion point therewith and/or to define intersection points with cortical bone on opposite sides of the bone.

In an exemplary embodiment of the invention, the vector is defined using a radiation source inside the bone, which illuminates the bone and the aperture in the implant, and optionally overlying soft-tissue. In an exemplary embodiment of the invention, the vector is defined by using two sensors, on opposite sides of the bone, to define a ray intersecting the light source and the bone (e.g., and an aperture in an implant) and along which the tool guide may be aligned. Optionally, the light source is placed within a cannulation of an intramedullary nail. In general, in an exemplary embodiment of the invention, the aiming is according to the location of the nail and the hole in the nail, while the desired result is to correctly lock the nail to a bone via correctly placed locking elements. In addition, it is noted that the overlying soft tissue may interfere with the ability to accurately locate the hole. In an exemplary embodiment of the invention, the sensor is pressed against the soft tissue, thereby reducing the soft tissue thickness and/or improving signal collection therefrom.

Alternatively, trans-illumination may be used, with the light source on one side of the bone, passing through the bone and aperture in implant and reaching a sensor on an opposite side of the bone.

In an exemplary embodiment of the invention, one or more sensors are provided on a tool guide that can be advanced into the patient to contact the bone.

In an exemplary embodiment of the invention, the sensor includes an array of sensors which can be used to indicate an estimated center point of an exit of a ray from said light source from a skin of the patient. Optionally, the array includes a plurality of discrete sensors with an aperture there-between for passage of said tool between. Optionally or alternatively, the array is a two dimensional array providing a two dimensional representation of light intensity at different points overlying the bone.

In an exemplary embodiment of the invention, a targeting system is included with a display indicating a desired drilling location and/or orientation.

An aspect of some embodiments of the invention relates to a targeting system including at least one optical element for sensing or generating radiation, rigidly coupled to a movable tool guide. In an exemplary embodiment of the invention, the tool guide is arranged to move along towards a target area identified using the optical element. In an exemplary embodiment of the invention, the tool guide is adapted for insertion through soft tissue and to contact a bone. Optionally, such insertion uses a sharpened rod, cutting element and/or drill inserted through, along or on the tool guide.

In an exemplary embodiment of the invention, the tool guide includes one or more optical elements, such as radiation sources and/or radiation sensors useful in identifying the target area and/or for determining a desired correction in an aiming of said tool.

An aspect of some embodiments of the invention relates to identifying a target area in a bone by irradiating the bone and an implant with radiation and detecting an aperture in said implant, aligned with the bone, by reflection from one or more of the bone, implant or a separate reflector, for example, a reflector (e.g., instead of or in addition to a light source) is inserted into a bore in the implant and aligned with an opening in the implant.

An aspect of some embodiments of the invention relates to identifying a target area in a bone by irradiating the bone and an implant with radiation from outside the body and detecting the radiation after it passes through the bone and an aperture in the implant, using a sensor inside the body and/or using a sensor outside the body.

An aspect of some embodiments of the invention relates to identifying an aperture in an implant and/or bone, by scanning the bone using trans-illumination of the bone and/or implant. Optionally, the scanning includes moving one or both of a sensor and a radiation source. Optionally or alternatively, the scanning includes using a sensor array and/or a radiation array. Optionally, scanning is electronic.

In an exemplary embodiment of the invention, scanning is used to detect both an axial (e.g., relative to bone and/or implant) location of the aperture and a transaxial location. Optionally, the scanning is used to identify a general layout of the bone, so that a tool guide can be aimed to transect a bone through its axis.

In an exemplary embodiment of the invention, an additional sensor is used to detect at least an approximate location of the implant and/or bone.

An aspect of some embodiments of the invention relates to a guiding a tool to a bone using a sensor and radiation source, in which one or both of the sensor and radiation source are moved out of the way and a tool guide taking their place, for guiding the tool after a desired target area is identified.

It should be noted that in some embodiments, a single sensor arrangement and single source are used, with one inside the body. However, this may be less preferred than using two fixed points outside the body, as the accuracy of any defined vector may not be as good.

An aspect of some embodiments of the present invention relates to a surgical instrument (e.g., a targeting system), providing for accurate targeting of the location and orientation of the holes intended for screw insertion, in an intramedullary nail. The device comprises, in general, radiation source, one or more arrays, such as matrix(s) of sensors, and a processing unit, and optionally provides for the attachment of additional surgical instruments such as a drill sleeve and\or a drill.

In an embodiment of the present invention the radiation (also referred to as “light”) source comprises an electromagnetic radiation emitter, an electrical power source (including for example, but not limited to, a battery, or an external power supply), and the means to connect the electromagnetic radiation emitter and the electrical power source, for example a cable.

In an embodiment of the present invention the emitting component is placed within a cannulation of the intramedullary nail, along its long axis. In an exemplary embodiment of the present invention said emitting component is embedded within an elongate element, for example a tube using, for example, but not limited to, epoxy.

In an embodiment of the present invention said tube can be inserted into the cannulation of an intramedullary nail, and can be advanced along said cannulation to reach the area of the distal nail holes. Optionally, the elongate element is flexible enough to bend with a bend in the cannulation, if any.

In an embodiment of the present invention the tube is marked at one or more longitudinal positions (e.g., at its proximal end) to indicate relative location of the emitting components and the tube, for example, by the marks being aligned with a proximal end of the nail. This allows at least initial alignment of the emitting components with the nail holes at the nail distal end and/or with any external detector.

In an embodiment of the present invention the tube of the light source is marked to indicate distance of the mark from the emitting component. This enables alignment of the emitting component with the nail holes at the nail distal end.

In an embodiment of the present invention the tube of the light source includes one or more protrusions which interfere with the apertures in the nail and thereby allow better relative positioning thereof. In an exemplary embodiment of the invention, such protrusions comprise perpendicular spines and/or a resilient protruding band. Optionally, the outer diameter of the insert at the protrusions is slightly larger than the inner diameter of the nail cannulation, so that the light source will get stuck when passing by a hole, but can still be pushed past such a hole, due to the elasticity of the protrusions. Optionally, the axial extent of the protrusion is selected to match the hole diameter, so that the insert is held snugly in place.

In another embodiment of the present invention the emitting component is placed outside the treated extremity, close to the skin. In an exemplary embodiment of the present invention said emitting component is embedded within an enclosure.

In an embodiment of the present invention such tube or enclosure (“housing”) is made of opaque material. Such materials include, for example, without limitation, stainless steel and polymers. Such enclosure many have openings made of translucent material against the location of the emitting component. Such translucent materials include, for example, without limitation, polymers, selected to allow the passage of radiation at specific radiation bands.

In an embodiment of the present invention the emitting component and/or its housing are designed for a single use. In another embodiment of the present invention the emitting component and/or its housing are designed for multiple uses.

In some embodiments of the present invention, when the emitting component is placed outside the treated extremity, the light emitting component and sensor are placed parallel to each other. Where said emitting component is placed outside the treated extremity, and sensor optionally comprises sensors placed on a matrix, at least 3 sensors are incorporated into a sensors-matrix. In an embodiment of the present invention, the emitting component and the sensor (forming an “emitter-sensor-assembly”) are located such that the treated extremity is placed between them. The emitting component and sensor are mounted on a structure that keeps the emitting component and sensor matrix parallel, and/or allows or urges the component and/or sensor to return to a parallel position. In another embodiment of the present invention the emitting component and sensor matrix are placed on the same side of the treated extremity (for example, when detection of reflected energy returned from the treated extremity is desired). In another embodiment of the present invention the emitting component is placed on one side of the treated extremity with sensor matrices located on both sides of the treated extremity—one adjacent the emitting component and the other one parallel it, on the other side of the treated extremity. In another embodiment of the present invention both emitting components and sensor matrices are located on both sides of the emitter-sensor-assembly, providing for illumination of the nail hole from both its sides and for sensors located on both hole sides.

In some embodiments of the present invention the distance between said sensor matrices (of “sensors-assembly”) or emitting component and sensor matrix (of “emitter-sensor-assembly”) is adjustable.

In an embodiment of the present invention, once nail hole location and orientation are successfully detected, the sensors matrix(s) is located such that the line passing through its geometrical center and perpendicular to its surface coincides with the long axis of the detected nail hole.

In an embodiment of the present invention a surgical tool (e.g., drill sleeve) is connected to at least one sensors-matrix of said sensors-assembly or emitter-sensor-assembly, such that the long axis of the drill sleeve coincides with the geometrical center of the matrix. Optionally, this means that the long axis of the drill sleeve coincides, once proper targeting is achieved, with a central axis of the hole in the nail In some embodiments of the present invention, said drill sleeve is combined into the enclosure of the emitting component.

In some embodiments of the present invention, said drill sleeve may be connected to any of the components of said emitter-sensor-assembly in a manner such that the drill sleeve takes the place of the emitting component/sensors matrix, after proper location of the nail hole.

In an embodiment of the present invention said drill sleeve is fixed to said sensors-assembly or emitter-sensor-assembly. In another embodiment of the present invention said drill sleeve can be moved, along its long axis, relative to said sensors matrix. In an exemplary embodiment said drill sleeve can be moved so that its distal end is placed against the bone (inside the body) after proper targeting is carried and an incision to the treated extremity is made. In another embodiment of the present invention said drill sleeve can be moved horizontally relative to the emitting component and sensor.

In an exemplary embodiment of the present invention the emitting component and/or sensor are replaced, following proper positioning of the nail hole, with the drill sleeve. Such position change can be performed, for example, automatically or free-hand (e.g., using information provided by camera(s), or by some type of accelerometer(s), or gyroscope, or compass, or hall sensor connected to the emitting component and/or sensor matrix and/or drill sleeve, or a combination of the above with a set reference point in space; camera(s), accelerometer, gyroscope, compass, hall sensor are defined hereinafter as “location aid unit”).

In some embodiments of the present invention said drill sleeve incorporates sensors into its perimeter at its distal and/or proximal end. In an exemplary embodiment of this invention the number of sensors placed on the drill sleeve perimeter is at least 3. In an embodiment of the present invention said sensors are exposed to the light emitted from the emitting component via light guides placed within or outside the wall of the drill sleeve. In another embodiment of the present invention certain parts of the drill sleeve, located beneath the sensors, are made of light conductive material (for example, but not limited to, polycarbonate). In an exemplary embodiment of the invention, location circuitry preferentially uses light detected by these sensors over external sensors, if relevant.

In an embodiment of the present invention said sensors are connected to a display. In an exemplary embodiment said display is located at said drill sleeve proximal end.

In an embodiment of the present invention said drill sleeve is used without additional sensors located outside the body (i.e., without the sensors-assembly or sensors of the emitter-sensor-assembly). In such an example, the number of sensors placed on the drill sleeve perimeter is optionally at least 3.

In some embodiments of the invention, the emitting component (e.g., laser-based), the sensor matrix, and some type of location aid unit(s) are connected to a power drill (for example, but not limited to, an off-the-shelf power drill). The emitting component and sensor matrix (emitter-sensor-assembly) provide for location of the intramedullary nail hole and for its orientation. Once correct drilling position and orientation is located the power drill is moved, based on information from the attached location aid unit(s) (and, optionally, its combination with a set point in space) until the drill bit connected to the power drill is positioned against the location desired for drilling, at the correct orientation.

In an embodiment of the present invention said sensors-assembly or emitter-sensor-assembly is connected to a tripod-like device, to enable stable positioning and operation. In another embodiment of the present invention said sensors-assembly or emitter-sensor-assembly is provided with means for connection to the operation bed or cart, for example, one or more rods, cables, straps and/or buckles. In another embodiment of the present invention said sensors-assembly or emitter-sensor-assembly is hand-held.

In an embodiment of the present invention means are provided with the system (emitters and sensors) to block background radiation from interfering with radiation emitted from emitting component. In an exemplary embodiment of the present invention an opaque fabric sleeve is provided.

In an embodiment of the present invention said sensors-assembly or emitter-sensor-assembly enables movement of the drill sleeve along its long axis after proper targeting is carried and an incision to the treated extremity is made. In an embodiment of the present invention said movement is carried manually. In another embodiment of the present invention said movement is carried automatically.

In an embodiment of the present invention such sensors-assembly or emitter-sensor-assembly is placed against the skin of the patient (in contact with the skin). In another embodiment of the inventions such sensors-assembly or emitter-sensor-assembly is not placed in contact with the patient skin.

An aspect of some embodiments of the present invention relates to imaging of body anatomies (including, but not limited to, bones, and soft tissue) for the detection of abnormalities in different tissues and/or implants and/or apertures in implants. It is noted that, generally, implants have a different transparency from tissue and may block light much more effectively than tissue, have a different wavelength-dependent absorption profile. A difference in texture of the target anatomy results in change in transmission, reflection, and absorption characteristics of radiation directed at said anatomy.

In an exemplary embodiment of the invention, when a targeting system as described herein is moved along a bone or traverse to a bone and/or relative to apertures in an implant, variation in sensed light is produced. In some embodiments, electrical scanning (e.g., of an array light source and/or an array detector) rather than physical movement, is used.

In an exemplary embodiment of the present invention said imaging is used for the detection of fractures in bones (for example, but not limited to, hand and foot bones). In another exemplary embodiment of the present invention said imaging is used for the detection of any changes in the structure or texture of tissues (either hard or soft tissues).

In an embodiment of the present invention said imaging is performed using an emitter-sensor-assembly, similar to the one described above. The emitting component(s) (placed either on one side of the involved anatomy or on both its sides) provides said pulsed radiation, which is directed at the anatomy. Certain amount of the energy is absorbed by the illuminated tissues while some of the energy is reflected by the tissues in the target anatomy, and some of the energy is transmitted through the entire target anatomy. The sensors matrix is placed either on the same side of the illuminated anatomy as the emitting component (detecting reflected energy), or on the other side of the illuminated anatomy (e.g., parallel to the emitting component, detecting transmitted energy), or on both sides of the illuminated anatomy—adjacent to the emitting component and parallel it, on the other side of the illuminated anatomy (detecting both reflected and transmitted energy). In an exemplary embodiment of the present invention both emitting component and sensors assembly are placed on both sides of the illuminated anatomy. In an embodiment of the present invention a surgical tool (e.g., biopsy needle) is connected to the emitter-sensor-assembly, in a similar manner to that described above for a drill sleeve. In some embodiments of the invention, the sensor is placed at several different angles and/or positions with respect to the source, and radiation is collected from different positions and/or angles and then processed to provide a spatial map.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Exemplary Light Source Designs

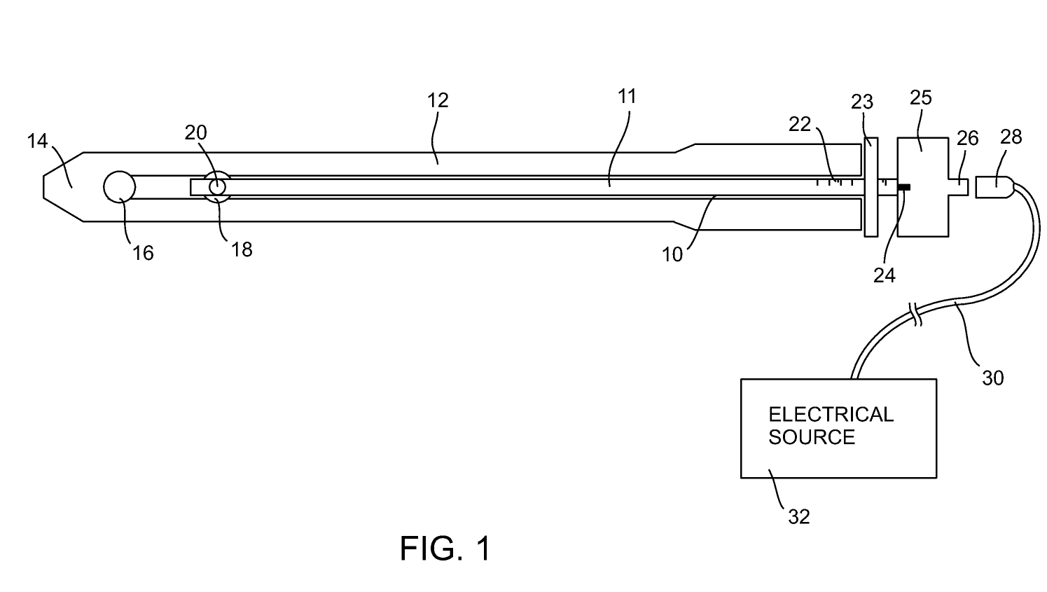

Referring now to the drawings, FIGS. 1, 2A-2C, and 3, illustrate exemplary light sources, in accordance with some embodiments of the invention.

FIG. 1 presents a light source 10 (e.g., providing for continuous or pulsed radiation) intended for use within a cannulation 14 of an intramedullary nail 12. In an exemplary embodiment of the invention, light source 10 comprises an elongated section 11 optionally made of opaque material, with a “window” 20 made of translucent material. An emitting component (for example, a LED, or the distal tip of a light guide, such as optical fiber) is located adjacent window 20, optionally embedded within elongated section 11 of light source 10. Window 20 is positioned adjacent one or more transverse apertures of the nail, for example apertures 16 and 18, which may be, for example, through holes. In use, when window 20 emits light, this light passes through apertures 16 (or 18) and is detected outside the body and used to help aim a tool at the bone overlying the hole and at the holes itself.

Optionally, light source 10 comprises a handle 25 for axial advancement along cannulation 14 and/or for rotation of source 10.

Optionally, light source 10 includes a connector 26 for an electrical source 32, for example, for providing power to the device. Alternatively, light may be provided from an external light source. In an exemplary embodiment of the invention, electrical source 32 is connected to light source connector 26 via a cable 30 and a connector 28. An electromagnetic radiation emitter component is located, optionally, at the distal tip of elongated section 11, against window 20 (e.g., in the case of LED). In this case electric wiring connecting said emitter to power source may be threaded through elongated section 11.

In an embodiment using an external light source, the electromagnetic radiation emitter is optionally located in handle 25. In this case a light guide transmitting radiation from emitter to window 20 is optionally provided within elongated section 11, with the light guide tip located at proximity to window 20. In an alternative design, an electromagnetic radiation emitter is located outside handle 25 or elongated section 11. Optionally, light source handle 25 and connector 26 include a cannulation through which a light guide of said emitter is threaded into elongated section 11 and/or otherwise define a light guiding channel. Optionally, the emitter connector is connected to connector 28 of electrical source 32.

In an exemplary embodiment of the invention, light source 10 may be marked along its elongated section with markings 22 indicating, for example, distance from the translucent window at the distal end of the light source and/or indicating when window 20 is adjacent a hole in the nail. This may allow light source 10 to be positioned so that light is emitted form a known hole.

In an exemplary embodiment of the invention, light source 10 is lockable in place and/or preventable from axial advancement and/or retraction. Optionally, a ring 23 may be provided to be placed at the location of desired distance and optionally engage elongate element 11 so that it cannot be advanced. Optionally, this is used to keep the light source in place once properly located against the hole of the nail. Optionally or alternatively, a locking element, optionally ring 23 itself, is selectively securable so as to lock elongated section 11 to nail 12. Optionally, handle 25 and/or elongate element 11 includes an orientation indication indicating the rotation of light source 10 (e.g., light sources thereof, relative to the nail, once placed within it.

In an alternative design, handle 25 has an aperture formed therein and allows elongate element 11 to be moved along it and selectively locked in place, for example, using a spring loaded clamp in handle 25 pressing thereon. Optionally or alternatively, handle 25 is configured to attach to a proximal side of the nail. Optionally, the nail includes a mechanism for attachment of handle 25 thereto, for example, a threading or a snap-type interfering element (e.g., protrusion and/or recess).

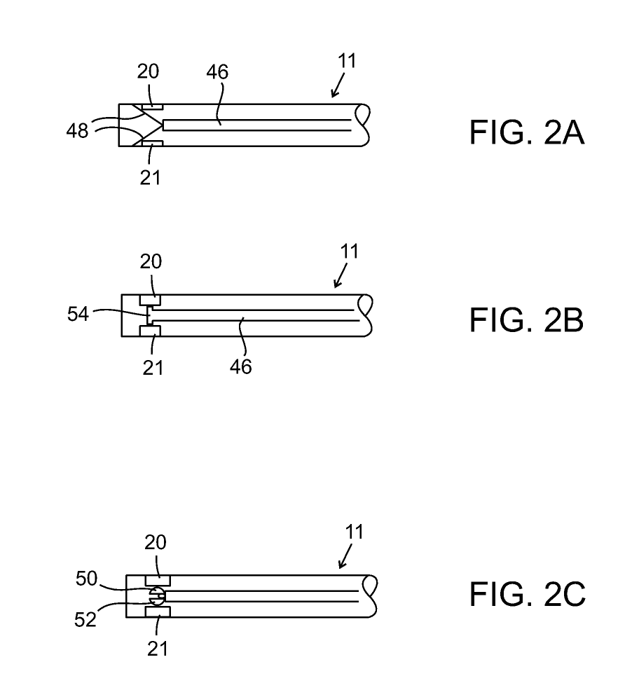

FIGS. 2A-2C present different exemplary designs of bi- or multi-directional emitting component at the distal end of light source 10. Optionally, elastically extending protrusions (or protrusions which can be extended by manual control (e.g., via a pneumatic input to a bladder at the protrusions or via a pushable stylet) may have light emitting sections, so as to ensure alignment of light emitting with the holes in the nail.

FIGS. 2A and 2B present a light transmitting element (for example optic fiber) 46 embedded within the elongated section 11 of light source 10. The light is radiated via windows 20 and 21, located, for example, 180 degrees apart on the perimeter of the distal section of the elongated section 11 of light source 10.

In FIG. 2A the light is diverted towards the translucent windows using reflective surfaces 48, which are optionally placed at an angle of about 45 degrees relative to the windows 20, 21 and the elongated section 11.

In FIG. 2B the light is diverted towards the translucent windows using directional couplers 54.

In an exemplary embodiment of the invention, all the light is aimed to exit out of a window and out of the implant, so as to reduce heating of the bone and/or other tissue by locally absorbed light.

FIG. 2C presents emitters 50 and 52 (for example LEDs) embedded within the elongated section 11 of light source 10. The light is radiated via windows 20 and 21, located 180 degrees apart on the perimeter of the distal section of the elongated section 11 of light source 10. In an exemplary embodiment of the invention, the light from 50 and 52 is emitted two rays at 180 degrees relative to each other.

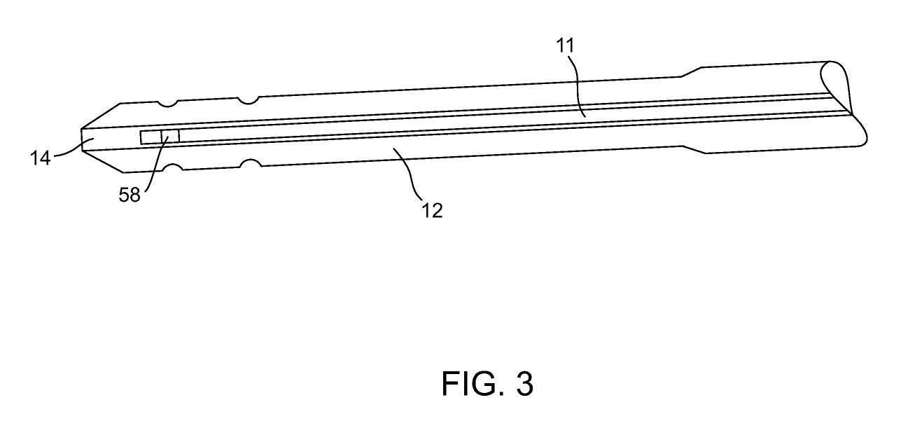

FIG. 3 presents an alternative design for distal section of light source 10, placed within cannulation 14 of intramedullary nail 12. In this embodiment translucent window 58 provides for 360 degrees illumination around the perimeter of elongated section 11 of light source 10. The light emitter (not shown) can be, for example, but not limited to, a LED radiating in 360 degrees, or the output of a light transmitting element, such as optical fiber placed against a conical mirror that disperses radiation in 360 degrees around the tip of the light transmitting element.

In an exemplary embodiment of the invention, elongate element 11 is rigid enough to be pushable and not collapse or fold inside cannulation 14. Optionally or alternatively, element 11 is rigid enough to transmit torque along its length and twist less than for example, 10 degrees, 5 degrees, or 1 degree. Optionally, however, elongate element 11 is made flexible enough so it can bend with the cannulation 14 is not straight. For example, a bending of, for example, a bending radius of 20 cm, may be provided for.

In an exemplary embodiment of the invention, elongate element is between 5 and 50 cm long, for example, supporting a distance of between 5 and 40 cm between the proximal end of an intramedullary nail and the location of the light emitting aperture in the nail. It is noted that when such distances are relatively long, jigs for controlling a drilling location relative to a nail distal hole are unwieldy and may be inaccurate.

In some embodiments, light source 10 includes a sensor at its tip, rather than a light source, with light being provided from outside the body. This may eb useful, for example, with thin bones and low thickness of overlying tissue, to compensate for the smaller collection area of such a sensor.

In an embodiment of the invention a k-wire with light emitting tip is used in order to emit light to the implant holes location or for other orthopedic uses. For example, k=2 and each wire is directed to different window. In another example, k=4, 6, 8 . . . , and different wavelength might be emitted by each one of wires k wires. In an exemplary embodiment of the invention, the structure of such a k-wire is a metal sheath surrounding an optical fiber, with one or more windows cut in the side of the sheath (and reflectors or diffusers provided in contact with or adjacent the fiber, to help light escape therefrom). An alternative design has the sheath surrounding electrical wires and one or more LEDs embedded within the sheath. Optionally, the sheath has a diameter suitable for use as a k-wire, for example, between 0.2 and 3 mm, for example about 1 mm, for example following standard sizes. Optionally, the sheath also allows plastic deformation of the k-wire.

In an exemplary embodiment of the invention, such an illuminating k-wire is used when guiding a drill to a repositioned bone segment, for example, which segment is mounted on the k-wire. In such an example, the segment is manipulated or held in place using a k-wire (with an open wound or a closed wound) and the drill is aimed at the illuminated portion to drill a hole in the bone segment. This may be useful for installing bone plate in complex fractures.

In an exemplary embodiment of the invention, the tip of the k-wire is formed as a circle or unclosed “C” shape with an aperture therein, which circle is light emitting (e.g., having a leaky optical fiber. This may be used, for example, as a target for aiming a drill or other tool towards the aperture.

Light Source Exemplary Properties

In some embodiments of the present invention the electromagnetic radiation emitter comprises a LED, or a number of LEDs. In another embodiment of the present invention the electromagnetic radiation emitter comprises a laser source. In some embodiments of the invention the electromagnetic radiation emitter comprises an arc lamp, a fluorescent lamp, and/or a gas discharge lamp (for example, but not limited to, xenon). In some embodiments, the light is generated outside the body and conveyed into the body using a rigid or a flexible light guide (e.g., a fiber optic).

In an exemplary embodiment of the invention, the radiation is continuous. In some embodiments, the radiation is pulsed. Using pulsed radiation, rather than continuous, might allow using higher energy without causing damage to nearby tissue. In an exemplary embodiment of the present invention said radiation has pulse length of the order of milliseconds (e.g., 1-400 msec) with duty cycle of, for example, 0.1 that optionally allows increasing peak power by an order of magnitude, and still stay within maximum average power allowed by medical regulation. Other exemplary duty cycles are between 0.001 and 0.8, for example, between 0.05 and 0.2. Alternatively, said radiation pulse length is of the order of microseconds (e.g., 1-330 μsec). Alternatively, said radiation pulses are of length smaller than microseconds (e.g., 0.01 to 0.5 msec). Alternatively, said radiation pulses length is have a varying duration and/or durations smaller, intermediate or longer than specifically listed herein.

A potential advantage of using pulsed radiation or modulated continuous radiation is that coherent detection can be used to detect the pulses and reduce the effects of noise. Optionally, a same circuitry is used to generate the light modulation and the detection. Optionally or alternatively, an input from the light source is used to provide an indication of the modulation to a detection circuit.

In an embodiment of the present invention the electromagnetic radiation emitter is located near pre-formed apertures (holes) of the nail. In another embodiment of the present invention the electromagnetic radiation emitter is placed away from the holes of the nail and the radiation is transmitted to the area of the holes (e.g., using a “light-transmitting element”/“light guide”). In an exemplary embodiment of the present invention the light is transferred from an electromagnetic radiation emitter placed away from the nail holes to the area of the holes using optical fibers. Optionally, the light guide is selected according to the wavelength used, for example, to reduce heating of the nail and/or bone.

In an embodiment of the present invention the light source includes more than one radiation-emitter or light guide tip (both referred to as “emitting component”), which can be located adjacent more than one nail hole simultaneously.

In an embodiment of the present invention the light source emits radiation with wavelength in the near infrared (IR) range (for example, but not limited to 500 nm to 1600 nm). In an exemplary embodiment of the present invention, the light emitter is a LED or another laser source with wavelength of 750 nm to 1200 nm. In another exemplary embodiment of the present invention, the light emitter is a LED or a laser source with wavelength of 940 nm to 980 nm. In another exemplary embodiment of the present invention, the light emitter is a LED or another laser source with wavelength of 1064 nm. Optionally, other wavelengths are used, for example, radio waves with very short wavelengths (e.g., tetra herz), which act light optical rays. In alternative embodiments, non-electromagnetic radiation (e.g., ionizing radiation) is used, of a type which is blocked by the implant but which passes through the body tissues (e.g., gamma radiation for steel implants. In some case photodiodes and/or CCD sensors can be used for such radiation as well.

In an exemplary embodiment of the invention, the wavelength (e.g., between 600 and 1200 nm) is chosen according to its ability to pass through bone and/or soft tissue, according to scattering properties and/or according to a level of expected noise from ambient light. In an exemplary embodiment of the invention, the wavelength is chosen according to a tradeoff between the detriment caused by scattering effects and the detriment caused by absorption effects. Thus, different wavelengths may be suitable for different tissue thicknesses and/or types.

In an embodiment of the present invention the emitting component emits a unidirectional light along a vector perpendicular to the long axis of the intramedullary nail (the “transverse plane”). In another embodiment of the present invention the emitting component provides for bi-directional light. The directions in which the light is emitted are at a relative angle of 180 degrees. Therefore, once placed within the nail, against the nail hole, the radiation emits from both sides of the nail hole, to form line comprising two opposing rays. In another embodiment of the present invention the emitting component provides light distribution in 360 degrees in said transverse plane, and the nail holes are used to direct the light. Optionally, for example, for oval holes, the light source is shaped so that the beam is narrower than the width of the hole, for example, being designed to indicate the hole center.

Optionally, for holes that are not perpendicular to the nail, the light source is designed (e.g., LED aiming direction, reflector placement) to project light in a direction along the hole axis, rather than perpendicular to the nail axis.

In an embodiment of the present invention the direction of radiation distributed from the emitting component is defined by the shape of the emitting component. In another embodiment of the present invention the direction of radiation distributed from the emitting component is set with the help of reflective surface(s) located within the housing of, or in close proximity to, the emitting component. In an exemplary embodiment of the present invention, where light transmission is by optical fibers, the direction of radiation distributed from the emitting component is defined using lens(s) and/or mirror(s) located in proximity to the distal end of the fiber.

In an exemplary embodiment of the invention, the beam is a narrow, substantially non-diverging beam. In another embodiment, the beam is a cone beam with an angle of, for example, between 3 and 30 degrees.

In another embodiment, two beams are used, with an outer ring being one wavelength and an inner section having another wavelength (or pulse coding). Then, an external detector can determine if most of the light reaching it is from the inner section or from the outer ring section, based on the encoding and/or wavelength. This may aid in targeting. However, it is noted that the vector used for inserting a locking screw may be selected to not be perpendicular to the bone.

It should be appreciated that the above properties may also be used with other light sources as described herein, other than light source 10.

Exemplary System Using Intramedullary Illumination

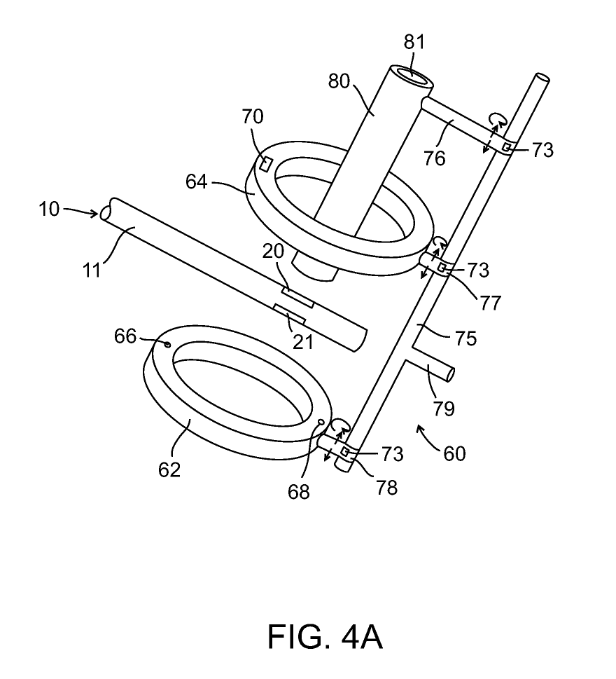

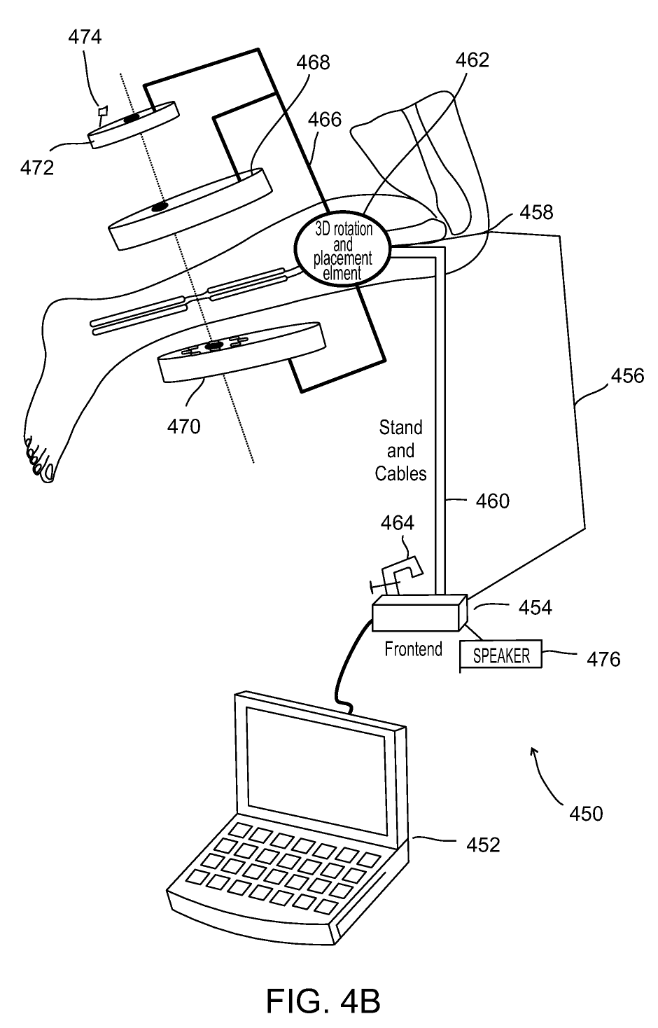

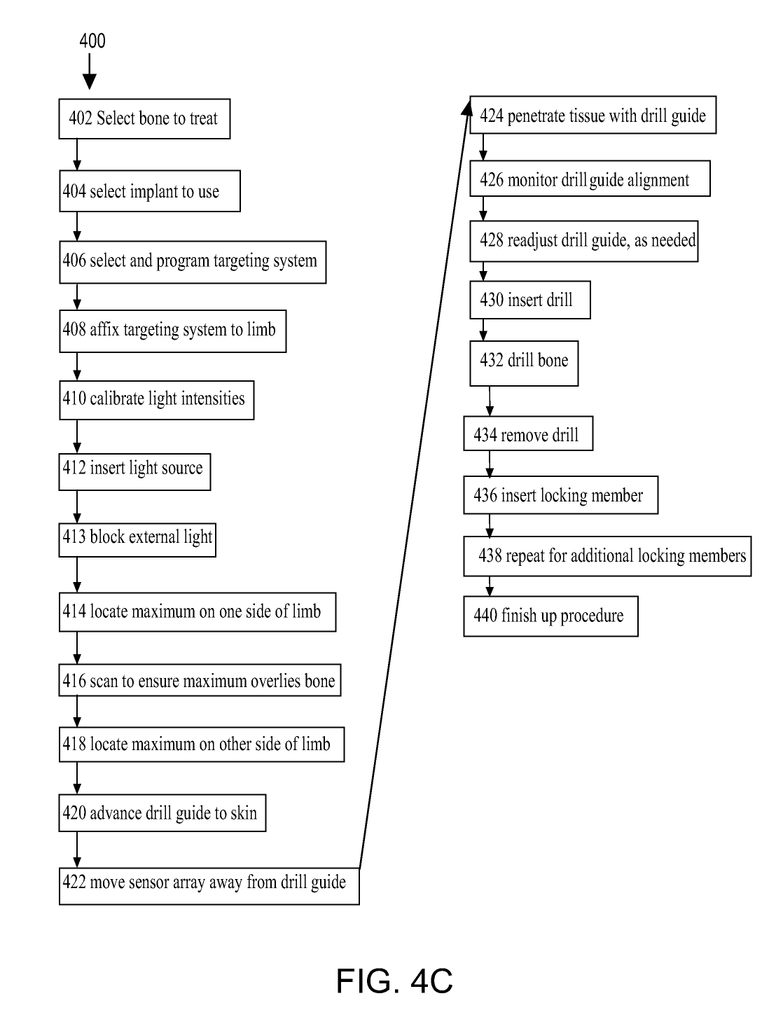

FIGS. 4A, 4B, 5 and 6 illustrate a targeting system including a sensors-assembly 60, intended for use in combination with a light source 10 placed within the cannulation of an intramedullary nail, in accordance with exemplary embodiments of the invention.

Sensors-assembly 60 comprises two sets of sensors, placed on two, optionally parallel and aligned, matrices 62 and 64. As noted herein, together with the light from windows 20 and 21, this defines a straight line (e.g., a vector) along which a tool is to be guided to the bone.

In an exemplary embodiment of the invention, the matrices are connected by a frame comprising rigid elements that provide for relative movement, for example as described below. In an exemplary embodiment of the invention, however, the parallel and alignment positions are known and easily reached (e.g., having snap-to settings or having a defining recess or depression).

In one exemplary embodiment, matrixes 62 and 64 are movable towards or away each other (e.g., at least one moves), for example, by one or both of connectors 77, 78 which connect them to a rigid frame 75 being slidable along the frame. Optionally, they can be locked in place, for example, using a screw or a spring-loaded clamp 73.

In an exemplary embodiment of the invention, such a clamp defines one or more predefined stop positions, to assist in returning elements to a desired alignment. Optionally or alternatively, one or both of matrixes 62 and 64 maybe moved out of the way, for example, by connectors 77 and/or 78 by connector being rotatable around rod 75.

In an exemplary embodiment of the invention, at least one of connectors 77 and 78 can slide along rod 75 to achieve the desired distance, according to the treated extremity, and can be locked once in the proper position, to avoid further movement.

In an exemplary embodiment of the invention, rod 75 is provided with a handle 79 used for holding the assembly. Alternatively (not shown), rod 75 may be provided with the option to attach to a tripod or a frame enabling attachment to a steady component (e.g., cart, table, treatment bed or patient body). For example, cables, rods and/or straps maybe used for such attachment.

In an embodiment of the present invention, when the emitting component is placed within the intramedullary nail, the sensors are organized in two sets, on two matrices, placed parallel to each other. The matrices are located such that the treated extremity is placed between the two matrices. The matrices are mounted on a structure (e.g., a frame) that keeps the matrices parallel (forming a “sensors-assembly”). In an exemplary embodiment two sensors are placed on each matrix. The sensors on each matrix are placed with a relative angle of 180 degrees between them. Alternatively, the sensors on one matrix are placed at a relative angle in the range of 1 to 179 degrees to the sensors of the other matrix. In another exemplary embodiment n sensors are placed on each matrix (n being at least 2), with a relative angle α between each two adjacent sensors, and a in the range of 1 to 361−n degrees. In another exemplary embodiment n is equal to 4 and the sensors are placed 90 degrees from each other.

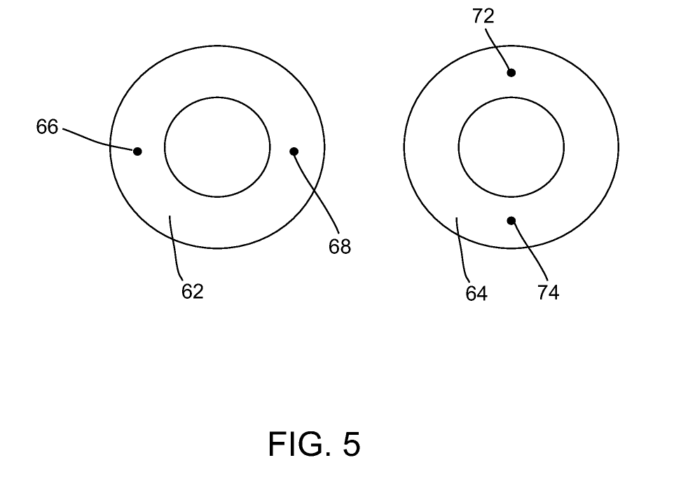

In this exemplary embodiment, as shown in FIG. 5, for example, matrices 62 and 64 each include two sensors each 66, 68 and 72, 74 respectively, located with an angle of 180 degrees between each two sensors on a single matrix. Matrices 62 and 64 are connected to the frame such that the sensors in each matrix are placed at an angle of α degrees (α ranging 1-179 degrees) to the sensors in the other matrix. Alternatively (not shown), the matrices may be of a different design (e.g., ring or surface, of different shapes) and may contain 2 or more sensors, arranged on their surface, or may be made of light sensing material. In an exemplary embodiment of the invention, the ring is completely covered with sensors and acts as an imager with a central aperture.

In an exemplary embodiment of the invention, during operation, matrices 62 and 64 are manipulated until they are located aligned with translucent windows 20 and 21 placed at the distal section of elongated section 11 of light source 10. Translucent windows 20 and 21 are located against the hole or holes of the intramedullary nail for which drilling is desired. Once sufficiently equivalent amount of radiation is detected by all sensors (e.g., 66, 68, 72, 74) on the two matrices 62 and 64, the long axis of the nail hole (e.g., a line connecting the circular shapes defined at the intersection of the hole and the nail surface) coincides with the line passing through the geometrical center of the matrices and perpendicular to the face of the matrices. Alternatively, when each matrix contains 3 sensors or more, a sufficiently equivalent amount of radiation shall be detected by all sensors on each matrix, but equivalence of detected radiation is not required between the two matrices. Optionally, the amount of light expected to be detected by each matrix is set depending on the bone being treated. A display (not shown) can be provided, for example on top of matrix 64, to guide the user as to the direction in which to move the assembly.

Referring to FIG. 4A, an optional indicator, for example visible indicator 70, is located, for example, on the top matrix 64. Indicator 70 turns on once a circuitry indicates correct location and/or alignment of the center of the nail hole. Alternatively, a set of indicators is provided, each turning on when a certain group of sensors (e.g., all sensors on a single matrix), detect sufficiently equivalent amount of radiation.

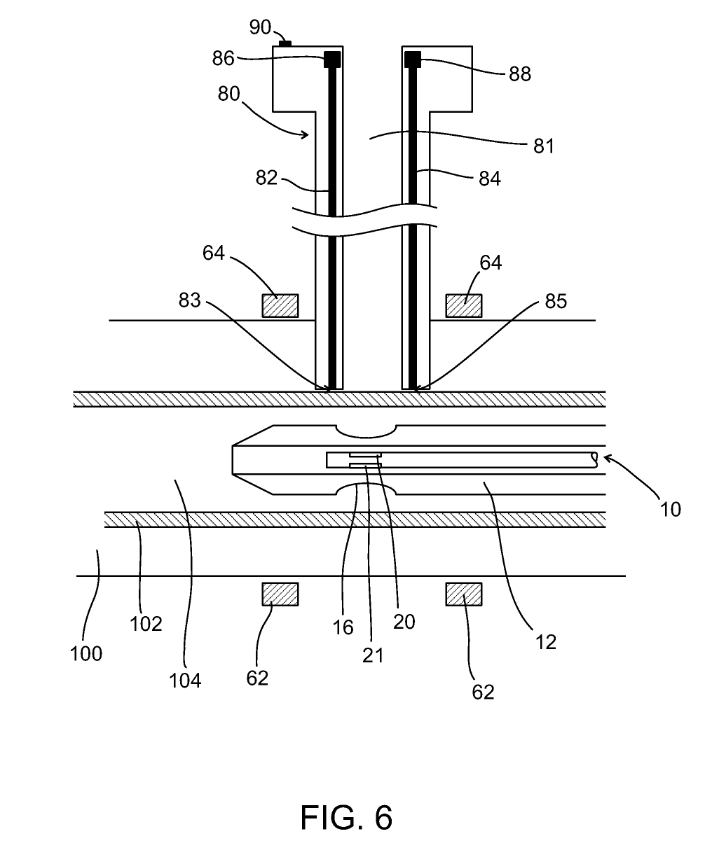

Referring now to FIG. 6, which shows an optional drill guide 80 usable in conjunction with the targeting system described herein, in accordance with some embodiment of the invention. In an exemplary embodiment of the invention, a drill sleeve 80 can be attached to frame rod 75, for example, using an arm 76. Optionally, arm 76 can move along rod 75 to advance drill sleeve 80 towards a bone 100 of an extremity, once the correct location and alignment of the nail hole is detected and an incision to the treated extremity is made. At this time, the long axis of drill sleeve 80 coincides with the line passing through the geometrical centers of matrices 62 and 64, and with the long axis of nail hole 16, creating a vector which connects 4 points.

In an exemplary embodiment of the invention, drill sleeve 80 has an insert, e.g., a sharpened rod, inserted therein to assist in advancing thereof. Optionally, sleeve 80 is cylindrical. Optionally or alternatively, sleeve 80 has an inner cross-section of a cone. Optionally or alternatively, sleeve 80 comprises a plurality of spaced apart rings.

In an exemplary embodiment of the invention, when it is desired to drill, a drill bit is inserted into drill sleeve 80 via a cannulation 81 thereof and/or a channel defined therealong. Alternatively other tools may be guided, for example, a self-tapping screw may be advanced along cannulation 81. Optionally or alternatively, a tool guide on which a tool rides may be used. For example, a locking element may be cannulated and travel along a thin rod which acts as guide 80.

In an embodiment of the present invention drill sleeve 80 is provided with one or more sensors (or inputs to radiation guides), such as sensors 86, 88, close to its proximal end. In an exemplary embodiment of the invention, at least 3 sensors are incorporated into the drill sleeve (one not shown). Sensors 86 and 88 (as well as any additional sensors provided) are connected to the distal end on the drill sleeve with light guides 82 and 84. Such light guides can be, for example, but not limited to, optical fibers, or channels made of light conducting material (for example, polycarbonate). Light conductors 82 and 84 (as well as any additional light conductors provided), are optionally placed against or terminate as translucent “windows” 83 and 85 (and any additional “windows” provided) at the distal surface of the drill sleeve.

In an exemplary embodiment of the invention, location circuitry (for example as described below) uses these sensors instead of or in addition to sensors 72, 74, as the drill guide is advanced into tissue and its distance to the nail is reduced and expected quality and/or quantity of detected light increases.

An optional visible indicator 90 is optionally located, for example, on the proximal area of drill sleeve 80. Optionally, indicator 90 turns on once circuitry indicates alignment with the location and/or orientation of the center of the nail hole, using the sensors 86 and 88 (as well as any additional sensors provided) available within the drill sleeve. A potential advantage of using sensors placed within the drill sleeve is to provide further assurance in correct placement and alignment of the drill sleeve with the nail hole prior to drilling. In some embodiments, proximity of sensors to the light emitter provides stronger signal and less background noise, potentially contributing to more accurate drill sleeve placement against nail hole. Electrical connections between the various sensors and the circuitry is not shown in the figure, for brevity.

In an embodiment of the present invention the sensors used for detection of the radiation emitted from the emitting component, are placed, for example, but not limited to, on an n-sided, simple, equiangular, equilateral polygonal, or on a circular, ring or surface (all referred to hereinafter as “matrix”). In an embodiment of the present invention the sensors are placed such that their distance from the center of the matrix is uniform. In an exemplary embodiment of the present invention the sensors are uniformly distributed on the perimeter of the matrix, providing for the same spatial angle between each two adjacent sensors and a line connecting the center of the matrix and the target area (e.g., bone, nail aperture). In an exemplary embodiment of this invention the number of sensors placed on the matrix is 2 or more, for example, 3, 4, 5 or more. In an exemplary embodiment of the present invention the sensors are photodiodes and/or array sensors such as CMOS or CCD arrays. In another embodiment of the present invention the entire matrix surface area (facing the detected radiation) is made of light sensing component, such as, but not limited to, CCD or CMOS, or a matrix of several diode sensors. In another embodiment of the present invention the sensors may be connected to an image processor. In an exemplary embodiment of the present invention the sensors may be connected to a power source.

FIG. 4B shows a complete targeting system 450, in accordance with an exemplary embodiment of the invention.

An optional controller 452, for example, a computer, with a display and keyboard, mouse and/or touchscreen input, maybe be used, for example, for programming the system and/or for displaying results and/or images. Circuitry 454 is shown as a stand alone box, which controls sensors and/or light sources. Optionally, circuitry 454 also serves as a stand, to which a frame 460 is optionally attached. An optional clamp 464 may fix the stand to a bed, for example.

As shown, circuitry 454 can provide power and/or control to a light source 458, via a cable 456. A frame 466 is optionally attached to frame 460 via a joint, for example, a 5- or 6-degrees of freedom joint, and includes a pair of sensor matrices 468 and 470 and an optional drill guide 472. An optional display 474 on guide 472 is shown.

Optionally or alternatively, an audio output 476, for example, for generating signals, including optionally speech sounds, such as instructions is provided.

In some alternative embodiments, light source 458 is a stand alone device. Optionally or alternatively, circuitry 454 may be integrated into frame 466 and/or sensor matrixes 468 and 470.

Exemplary Method of Use

FIG. 4C is a flowchart 400 of an exemplary method of treating a bone, in accordance with an exemplary embodiment of the invention.

Generally, during intramedullary nailing, once interlocking holes drilling is desired (especially to the distal holes of the nail), distal holes targeting is generally required. When using light source 10 (intended for placement within the intramedullary nail), it is inserted into cannulation 14 of nail 12, until the emitting component (translucent “windows” in the light source elongated segment (tube), for example windows 20 and 21, at the distal end of light source 10 are placed against the holes) is located against the involved nail holes where drilling is desired. Sensors-assembly 60 is placed such that matrices 62 and 64 are located on both sides of the treated extremity, against the approximate area of the holes. Sensors-assembly 60 is then slightly moved and rotated until the correct location and alignment of nail holes with the line passing through the geometric center of the matrices is achieved, as indicated on the assembly. Once the correct location and alignment are obtained, an incision is made to the skin at the area of the geometrical center of the matrix, the bone is exposed, and a drill sleeve 80 connected to sensors-assembly 60 is optionally advanced towards the bone. Drill sleeve 80 is optionally equipped with sensors and indicators similar to those provided on the sensors matrices, to provide for fine tuning of the required drilling location and orientation. Once the exact position for drilling is set, light source 10 is optionally pulled back, and drilling may commence via cannulation 81 of the drill sleeve. This procedure can be repeated for additional nail holes. Optionally, the most distal hole is drilled first.

In an exemplary embodiment of the invention, accuracy of drilling is to within 1-3 mm for a first hole and 1 or 2 mm for subsequent holes (e.g., relatively to the first hole), or better, for example, accuracies of within 1 mm. Optionally, the distance of the axis of the center of the drilled bore from the axis of the nail is between 0 and 3 mm, Optionally, less than 2 mm or 1 mm.

Referring specifically to FIG. 4C.

At 402, a bone to be treated is selected. Exemplary bones include, for example, hollow and/or long bones such as the Tibia, Femur and Humerus. It is noted that the described system can also be used on the outside of bones and for implants placed into trabecular portions of a bone.

At 404, an implant to be used is selected. While many of the examples herein relate to intramedullary nails, the methods described herein can be used for bone plates as well, where a correct alignment of a bone screw to apertures in the plate may be desired. A light source may be provided, for example, inside the underlying bone, or, for example, along the implant, for example, in a groove defined therein. Optionally, the implant is inserted into the body with a light source located adjacent or in a desired hole and once drilling and/or screwing is set up, the light source is pulled out, for example, being on a wire.

In an exemplary embodiment of the invention, the implant is a steel or titanium implant. Alternatively, the implant is formed of a composite material, such as carbon fibers and PEEK. It is noted that in such implants, x-ray imaging may not be sufficient to detect apertures and/or aperture orientation therein.

At 406 the targeting system to be used is optionally selected and/or programmed. For example, such programming may include one or more of expected absorption and/or scattering of light, expected accuracy, desired locking locations and/or orientations of apertures in an implant, effect of bone on light, a setting for different extremities and/or bones and/or implant, difference between detection at different parts along limb and/or sides of limb. Optionally, programming is by providing the system with one or more of a nail ID, patient ID and/or patient data.

At 408, the targeting system is optionally affixed to the limb being treated and/or otherwise coupled thereto or placed adjacent thereto. Optionally, the sensor matrixes are approximated to the skin of the extremity.

At 410 expected light intensities are optionally calibrated, for example, by trans-illuminating the extremity. This could be done, for example, by emitting a low intensity light, and increasing it until first indication of light is detected. This intensity could be used as a lower level intensity.

At 412, optionally after the implant is inserted, light source 210 is inserted into the nail (e.g., to a most distal locking hole to be used) and turned on.

At 413, light from external sources is optionally blocked, for example, using an opaque blanket (e.g., with a metallic layer) or a designated box.

At 414 one sensor matrix is moved to identify where there is a maximum light intensity and/or uniform for all sensors in the matrix. Optionally, a singe sensor is moved to identify the maximum. Optionally, a light guide is provided on the source and/or on the detector(s) to shape the transmitted and/or received beams. It is noted that an initial positioning by hand or using a jig may be quite accurate requiring only small corrections based on sensed light.

Optionally, the sensor(s) is pressed against the soft tissue, possibly increasing an efficiency of detection and/or reducing thickness thereof. Optionally, an optical coupling layer (e.g., a gel) is provided therebetween. Optionally, when an external light source is sued, a cooling layer, such as pre-cooled glass or a hollow transparent chamber with cold fluid inside is placed between the light source and the skin, to reduce risk of burning.

It is noted that the implant can often be inserted in several orientations. A planning stage of deciding what orientation to insert the implant in, which locking elements to use and which overlying soft tissue to go through, is optionally carried out, possibly at an earlier stage in the process. In an exemplary embodiment of the invention, the vector of anchoring member implantation is selected according to one or more of the following considerations: ability to locate hole via soft tissue, potential damage to overlying soft tissue and quality of anchoring of nail (e.g., damage to bone, correct mechanical results).

At 416, optionally during 414, it is ensured that the location is above bone. For example, when the matrix is moved along the bone and transverse to the bone, the maximum is expected to be found in a location surrounded by darker areas. Such an “image” may be collected pixel by pixel or it may be imaged, for example, using an imager.

At 418, a maximum and/or uniform illumination location is identified on an opposite side of the extremity, while maintaining the first found maximum on the first side of the extremity. It is noted that a straight line connects the two sensor matrices and the hole in the nail/implant. It is also noted that the matrices identify light as it is scattered and passes through the upper layer of tissue (e.g., skin).

At 420, a drill guide is optionally advanced to the skin.

At 422, a sensor array which interferes with the drill guide is optionally moved away. Optionally, the circuitry is configured to start using data from the sensors in the drill guide, for example, by detecting that the sensor matrix was moved.

At 424, tissue is penetrated with the drill guide. Optionally, an incision is made with a knife and a sharpened rod is inserted through the drill guide, to the bone, with the drill guide being advanced over the rod. Optionally, ultrasound or x-ray or other imaging methods are used to ensure that there are no major blood vessels, ligaments and/or nerves in the path of drilling.

At 426, for example, during insertion, the positional and/or orientational alignment of the drill guide is tested using the sensors thereon. If needed, the drill guide location and/or orientation are adjusted (428).

At 430 a drill (or other tool, such as a biopsy needle or self-taping screw) is inserted, if desired, and at 432 drilling is performed. In an exemplary embodiment of the invention, the use of a system as described herein avoids one or more of shaving of the nail by the drill, missing the bone with the drill and/or incorrect placement of the locking element.

At 434 the drill is removed and a locking member is optionally inserted (436).

At 438 the process (412, etc.) is optionally repeated for other drilling locations.

At 440, the procedure is completed, for example by suturing any open incisions.

Exemplary Determination of Correct Position and/or Orientation

In an embodiment of the present invention the signals captured by said sensor(s) on sensors-matrix(s) are processed by the circuitry to provide information of emitting component/sensors matrix and/or surgical tool location and orientation as compared to nail distal hole. In an exemplary embodiment of the invention, the processing comprises determining a vector interconnecting two optical elements outside the bone and the implant. Optionally or alternatively, the processing comprises separately identifying location and orientation at which the sensor signal is maximal and/or uniform for all sensors in the matrix. Optionally, uniformity is within 20%, 10% or better. Optionally, the level of uniformity used depends on the limb. In embodiments described below where both source and detector are outside of the bone, a single alignment with a maximum and/or uniform signal may be sufficient, if it is clear it passes through bone (e.g., 416).

In an embodiment of the present invention the information is displayed to the user. In an exemplary embodiment of the present invention the information is displayed using visual display. In some exemplary embodiments the information is presented using audible signal. In some exemplary embodiments the information is displayed using both visual and audible signals. In an embodiment of the present invention the display of information provides information as to the direction in which said sensors-assembly or emitter-sensor-assembly should be moved in order to arrive at a desired location and/or orientation in addition to or instead of an indication of the correctness of an instant location. In one example, the display comprises four lights around the circumference of the ring, indicating which direction to move the ring (3 being on might mean tilt from the plane of the ring) and/or their relative intensity indicating correct intensity.

In an embodiment of the present invention the circuitry adjusts radiation intensity (e.g., to ensure sufficient light reaching detectors and/or prevent over heating) and/or sets sensor sensitivity, for example, to ensure that the detected light is within a working range of the detector. Optionally, such settings are performed during calibration (e.g., 410). In an embodiment of the present invention where a location aid unit is used so that a tool guide can be positioned where a sensor detects the hole and the vector is aligned, during positioning process, the signals generated by said location aid unit (combined, optionally, with the location of a set point in space) are processed by the circuitry to provide information on spatial location. This may happen, for example, when the emitting component and/or sensor are replaced, following proper positioning of the nail hole, by the drill sleeve. In an embodiment of the present invention said information is displayed to the user.

In an embodiment of the present invention, nail hole location and orientation are successfully detected when at least 3 sensors located on a sensors matrix, or at least 4 sensors located on two, parallel sensors matrixes, or the surface of a sensor matrix made of light sensing component, detect sufficiently equivalent amount of radiation, providing for hole pattern.

Exemplary Detection Methods

In an exemplary embodiment of the invention, the illumination is detected using simple intensity detection. Optionally, light from outside, especially at the relevant wavelengths, is blocked. Optionally, the detectors have a narrow wavelength filter thereon and the source is a narrow wave band source matching said filter.

In an exemplary embodiment of the invention, coherent (synchronous) detection is used in which a detector modulation is matched to a source modulation.

In an exemplary embodiment of the invention, coherence-based detection is used, in which the detector only accepts photons with a correct coherence (phase), for example, using a same laser to illuminate both the bone and the detector and generate an interference therebetween.

In an exemplary embodiment of the invention, time of flight is used to detect light which is scattered less than other light. In this method, a time-of-flight measurement or temporal gating is used to reject light other than light which traveled a substantially straight line from the source to the detector, based on the time of flight of such light. This method may require tight control of distances and lengths of optical paths.

In an exemplary embodiment of the invention, the emitted light has a non-Gaussian power distribution, for example step-like cross-section of intensity. Optionally or alternatively, the emitted light has a high intensity in a circular band surrounding a darker center. This may allow to detect when a sensor is aimed at a side of an area of interest. Different colors and/or coding for different parts of the band may help in determining a correction detection. As can be appreciated, this can allow a hole to be detected with a single sensor.

Optionally or alternatively, that allows side illumination of area of interest.

Exemplary System Parameters

It should be noted that the numbers provided herein (and in other sections) are not necessarily limiting but may provide a guideline for use with some embodiments of the invention.

In an exemplary embodiment of the invention, the diameter of matrixes 62, 64 is between 3 and 20 cm, for example, between 5 and 12 cm. An exemplary diameter of an aperture in a matrix element is between 20% and 90% of the diameter thereof.

An exemplary length of the drill guide is between 1 and 10 cm, for example, between 4 and 6 cm. An exemplary thickness of the drill guide is between 0.1 and 3 mm.

In an exemplary embodiment of the invention, the distance between the sensor matrixes is settable to be between 3 and 45 cm, optionally with an accuracy of between 0.1 and 3 mm.

In an exemplary embodiment of the invention, light source 10 is between 5 and 50 cm long, for example, about 2-10 cm longer than the nail being used.

In an exemplary embodiment of the invention, the light intensity and detectors are set up to detect light through cortical bone of a thickness of, for example, between 0.2 and 3 mm and tissue of a thickness of between 1 and 20 cm, for example, between 5 and 15 cm.

Exemplary System Using External Trans-Illumination

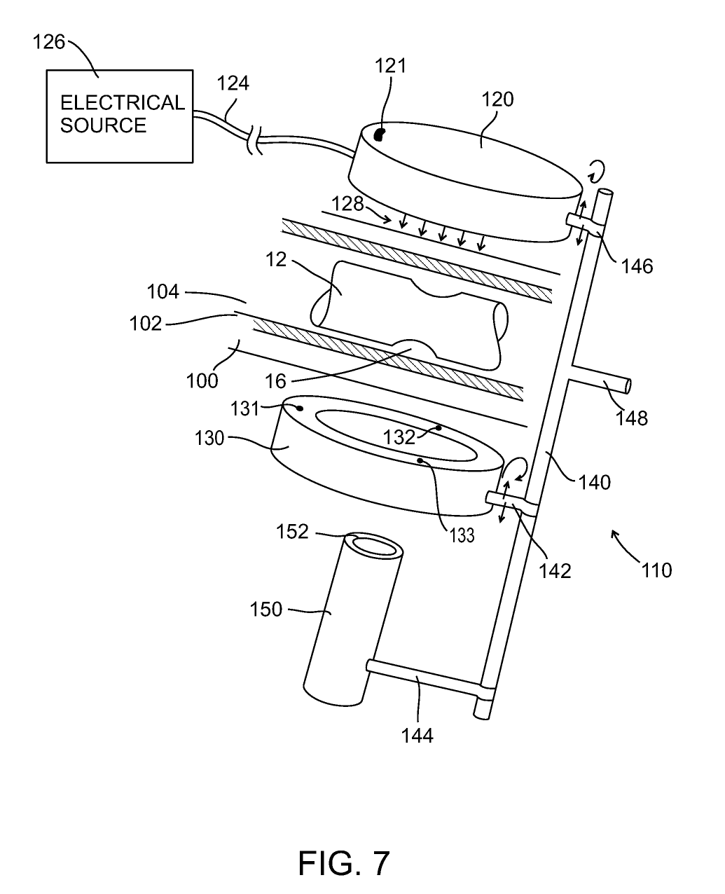

FIGS. 7, 8 and 9 illustrate additional exemplary designs of targeting systems including emitting component(s) and sensors matrix(s), in accordance with some embodiments of the present invention.

FIG. 7 illustrates a trans-illumination setup, in accordance with an exemplary embodiment of the invention, in which light is provided from one side of the bone, passes through soft tissue, cortical bone, aperture in nail, cortical bone, soft tissue and out to a detector.

In FIG. 7 an emitting component (within its housing) 120 is optionally connected to an electrical source 126 via a cable 124. Emitting component 120 emits radiation 128 towards treated extremity 100, optionally in the form of a tight beam or in the form of a first coded beam surrounded by or adjacent a second coded beam (so it can be determined which beam passed through the aperture). The emitting component(s) is, for example, intense pulsed light, such as a xenon flash lamp, or a laser source. The skin of the patient is optionally cooled (e.g., with a transparent cold element) or pre-cooled to prevent heat damage.

In an exemplary embodiment of the invention, a sensor matrix 130 is placed parallel and aligned to emitting component 120. The matrix(s) could also be placed with other relative orientations and/or relative positions, for example, to allow trans-illuminated or reflected light to arrive from different locations. Matrix 130 and component 120 are optionally connected by a rigid frame, for example, comprising a rod 140 and a set of one or more arms e.g., 142, 144, and 146. Exemplary optional movement directions of such arms is shown by arrows. An exemplary design of sensor matrix 130 is a matrix with 3 sensors 131, 132, 133 arranged at 120 degrees to each other. Another exemplary design (not shown) is a surface made of light sensing material. It should be appreciated that the sensor matrix may be of different designs, and of different shapes, and may contain a different number of sensors.

In an exemplary embodiment of the invention, emitting component 120 and sensor matrix 130 are placed outside the treated extremity 100. Intramedullary nail 12 is placed within the medullary canal 104. Emitting component 120 and sensor matrix 130 are placed against the skin at the area of the nail distal holes (for example, hole 16). Alternatively, sensor matrix 130 is placed such that it touches the skin, while emitting component 120 might be placed at a certain distance from the skin, to prevent over heating of skin area. Radiation 128 (either continuous or pulsed) emitted from emitting component 120 travels through treated extremity 100, bone cortex 102, intramedullary canal 104, and nail 12, and especially through nail hole 16. The wall of nail 12 is opaque to the involved radiation. The radiation detected by sensor matrix 130 creates a pattern of the nail hole(s) on the sensor matrix. This pattern may include an illuminated area for the light passing around the bone, surrounding a dark area, for light blocked by the nail, surrounding a light area of intermediate lightness, for light passing through the nail aperture. Multiple such patterns may be provided for different bail apertures. As described below, light collected from multiple locations may be used to generate a map or image of probable hole locations.

During operation emitting component 120 and sensor matrix 130 are placed adjacent the approximate location of the nail hole (e.g., hole 16) for which drilling is desired. Once all sensors of sensor matrix 130 (e.g., 131, 132, 133) detect a hole pattern (e.g., illuminated area surrounded by dark area) of sufficiently equivalent intensity, the long axis of the nail hole coincides with the line passing through the geometrical center of the sensor, and is transverse to the sensor plane.

Optionally, in this or other embodiments, the detectors are collimated towards an expected location of the hole in the nail.

An optional indicator, for example, visible indicator 121, is located, for example, on the top of emitting component 120. Indicator 121 turns on once circuitry indicates location and alignment of the center of the nail hole. A display (not shown) can be provided, for example on top of emitter 120, to guide the user as to the direction in which to move the assembly.

In the exemplary design of emitter-sensor-assembly 110 illustrated in FIG. 7, sensor matrix 130 is connected to rod 140 of the frame, via arm 142, and emitter 120 is connected to rod 140 via arm 146. Optionally, at least one of arms 142 and 146 can slide along rod 140, to achieve the desired distance between the emitter and sensor, and can be locked once in proper position. Optionally, rod 140 is equipped with a handle 148 used for holding the assembly. Alternatively (not shown), arm 140 may be provided with the option to attach to a tripod or a frame enabling attachment to a steady component (e.g., cart, table, treatment bed).

An optional drill sleeve 150, with cannulation 152 for drill bit insertion, can be connected via arm 144 to rod 140. Arm 144 can move along rod 140. After correct location and alignment of the nail hole is detected using sensor matrix 130, and an incision to the treated extremity is made, drill sleeve 150 is optionally advanced towards bone extremity 100 so that drill sleeve 150 will be placed against desired nail hole (e.g., nail hole 16), in a way that its long axis coincides with the long axis of the nail hole.

An exemplary design of drill sleeve 150 includes a design as described for drill sleeve 80, illustrated in FIG. 6, incorporating, for example, sensors, light guides and a visible indicator.

Exemplary Reflection Based System

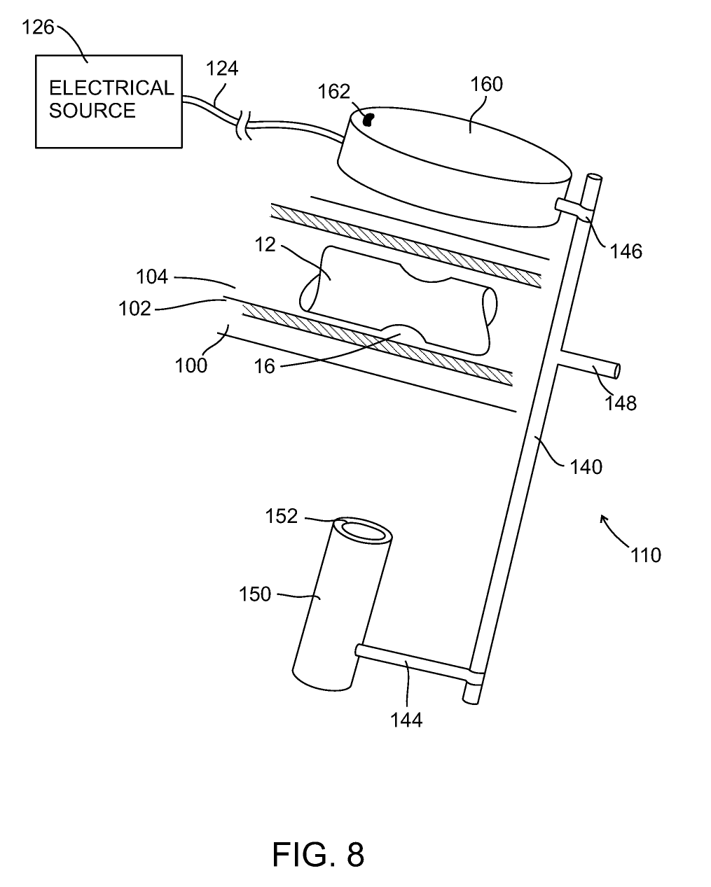

FIG. 8 shows a design similar to that described in FIG. 7, in which emitting component 120 is replaced by a combined emitter-sensor element 160. Element 160 combines emitting component(s), for example laser-light source(s), and sensors or a surface made of light-sensing material. In this embodiment radiation 128 (either continuous or pulsed) emitted from emitter-sensor element 160 travels through treated extremity 100, bone cortex 102, intramedullary canal 104, and nail 12, and especially through nail hole 16. The energy reflected from the bone and nail is detected by the sensor incorporated into emitter-sensor element 160, creating a pattern of the nail hole(s) on the sensor. Optionally or alternatively, a reflector is placed in the nail to reflect light at the aperture. Optionally or alternatively, the nail is selected or coated with a material having a higher reflectance at the wavelength used by the system shown in FIG. 8.

Alternatively, another sensor matrix (e.g., sensor matrix 130 as in FIG. 7 (not shown in FIG. 8)) can be added to the system, so that the emitter-sensor-assembly is similar to the system presented in FIG. 7, with element 120 replaced by element 160. The radiation passing through hole 16 and detected by sensor matrix 130 creates a pattern of the nail hole on the sensor. Optionally, the pattern is generated by moving (e.g., manually and/or under electronic control) the radiation source and\or the detector. Especially when the source light is relatively narrow, it may be desirable to change the projection of the light, with or without move of the detectors. Optionally, the pattern is visualized by the operator/physician or built up as an image using a processor unit. It is noted that, in general, the detected light levels and/or wavelengths are not suitable for unaided human detection and the systems described herein can provide such aid and/or creation of a usable targeting map/image.

In an exemplary embodiment of the invention, emitter-sensor element 160 (and sensor matrix 130, if provided) is placed outside the treated extremity 100. Intramedullary nail 12 is placed within the medullary canal 104. Optionally, emitter-sensor element 160 (and sensor matrix 130, if provided) may be placed against the skin at the area of the nail distal holes (for example, hole 16). Alternatively, emitter-sensor element 160 is structured such that the emitting component is placed at a certain distance (e.g., 3-8 mm) from the skin, to prevent over heating of skin area, while the sensors may be closer to the skin (e.g., 0-4 mm). If possible, pressing the skin is performed in order to allow better detection of the light. When the soft-tissue is presses, the light travels shorter distance, and the detectors are closed to the skin. The press is performed in the area of the holes, therefore in a case of nail implant, the broken bone is not located it the pressing area.

In an exemplary mode of operation emitter-sensor element 160 (and sensor matrix 130, if provided) is placed against the approximate location of the nail hole (e.g., hole 16) for which drilling is desired. Once sensors of emitter-sensor element 160 detect a hole pattern (e.g., darker area surrounded by illuminated area) of optionally a sufficiently equivalent intensity, the long axis of the nail hole coincides with the line passing through the geometrical center of the sensor, and is transverse to the sensor plane. In one example, the imager shows a dark line along where the nail is underlying, with bright areas overlying holes and outside of the nail. The difference in shape between the two areas can be identified, for example, manually or automatically. n case sensor matrix such as sensor matrix 130 is also combined into the system, data from both sensor elements—the sensor of emitter-sensor element 160 and sensor matrix 130, can be used to define a vector interconnecting the two sensors and the nail hole, used for the detection decision.

An optional indicator, for example, a visible indicator 162, is located, for example, on the top of emitter-sensor element 160. Indicator 162 turns on once an algorithm indicates location and alignment of the center of the nail hole. A display (not shown) can be provided, for example on top of emitter-sensor element 160, for example, to guide the user as to the direction in which to move the assembly.

In the exemplary design of emitter-sensor-assembly 110 illustrated in FIG. 8, emitter-sensor element 160 is connected to a rod 140 via arm 146. Arm 146 can optionally slide along rod 140, to achieve a desired distance, and can optionally be locked once in proper position. Rod 140 is optionally equipped with a handle 148 used for holding the assembly. Alternatively (not shown), rod 140 may include a coupling to provide an option to attach to a tripod or a frame enabling attachment to a steady component (e.g., cart, table, treatment bed).

In an exemplary embodiment of the invention, an optional drill sleeve 150, with cannulation 152 for drill bit insertion, can be connected via an optional arm 144 to rod 140. Optionally, arm 144 can move along rod 140. After correct location and alignment, the nail hole is detected using the sensor of emitter-sensor element 160 (and calculation circuitry), and an incision to the treated extremity is optionally made, drill sleeve 150 is optionally advanced towards bone extremity 100 so that drill sleeve 150 will be placed against desired nail hole (e.g., nail hole 16), in a way that its long axis coincides with the long axis of the nail hole.

An exemplary design of drill sleeve 150 provides for design as that of drill sleeve 80, illustrated in FIG. 6, incorporating sensors, light guides and a visible indicator. In such a configuration, drill 150 can take the place of sensor matrix 130 as described above. It is noted that some embodiments use simultaneous transmission and reflection modes to detect the nail hole positions.

Alternative Exemplary Reflection Based System

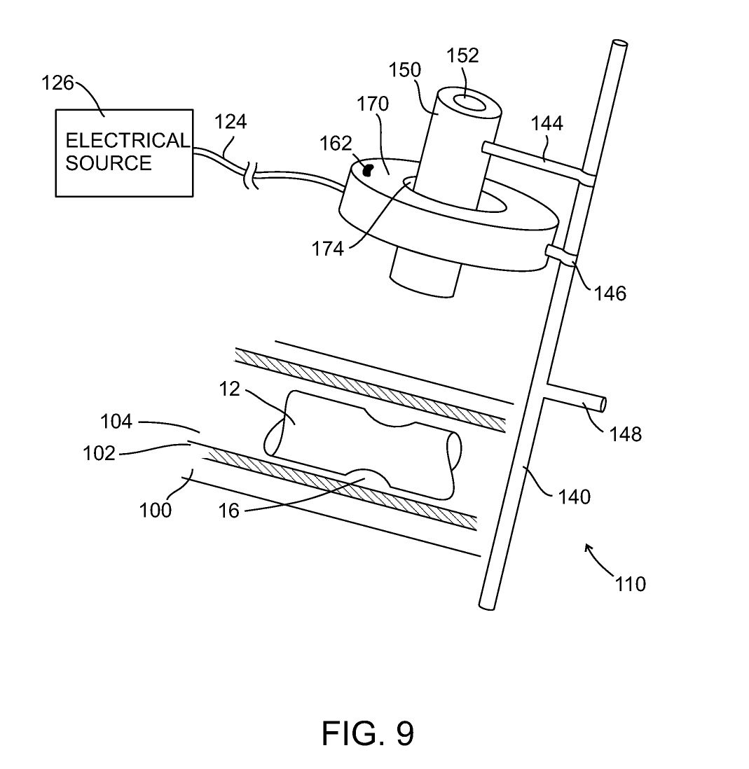

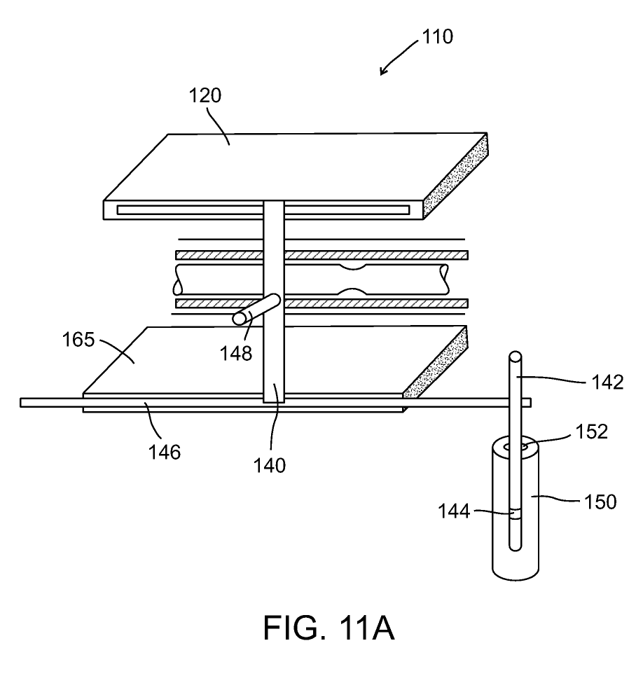

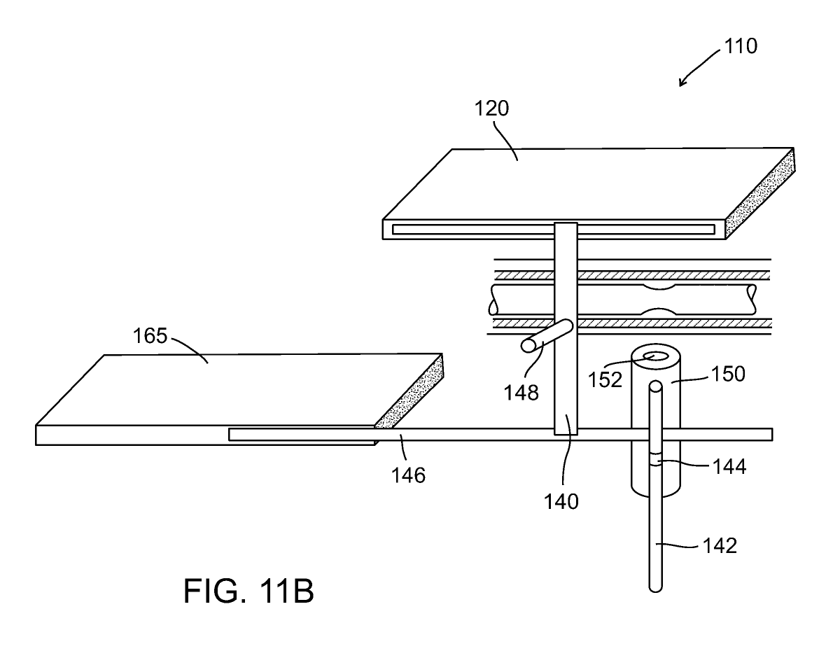

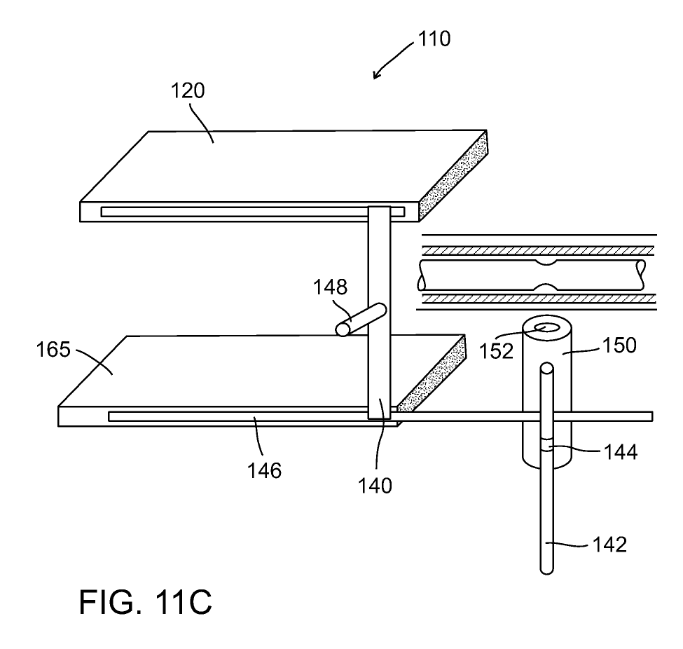

FIG. 9 shows an alternative design, similar to the design described in FIG. 8, except that an emitter-sensor element 170 (or an emitter) is provided with a hole 174 through which a drill sleeve 150 can be moved towards the bone following nail hole location and/or used as a sensor. Alternatively (not shown), the emitting component incorporated into emitter-sensor element 170 is located on a shutter, covering hole 174, which moves following location and alignment of the nail hole, and prior to advancing the drill sleeve through hole 174. The operation of emitter-sensor-assembly 110 illustrated in FIG. 9, is optionally the same as the operation described for the assembly presented in FIG. 8 (including the optional addition of sensor matrix, such as sensor matrix 130 as in FIG. 7, or sensor matrix 165 as in the later described FIG. 11, parallel and aligned to emitter-sensor element 170 and connected to rod 140 on the opposite side of the treated extremity).

In an exemplary embodiment of the invention, for example, in combination with reflective, trans-illuminating or transmissive embodiments, an additional sensor for gross alignments is used. For example, an eddy current-based sensor can be used to detect an approximate location of a hole in a steel implant, based on a change in a magnetic field created by induced eddy currents. Optionally or alternatively, a magnet maybe used to provide initial approximate location, for example, using a hall sensor on the sensor matrix.

Exemplary Non-Apertured System

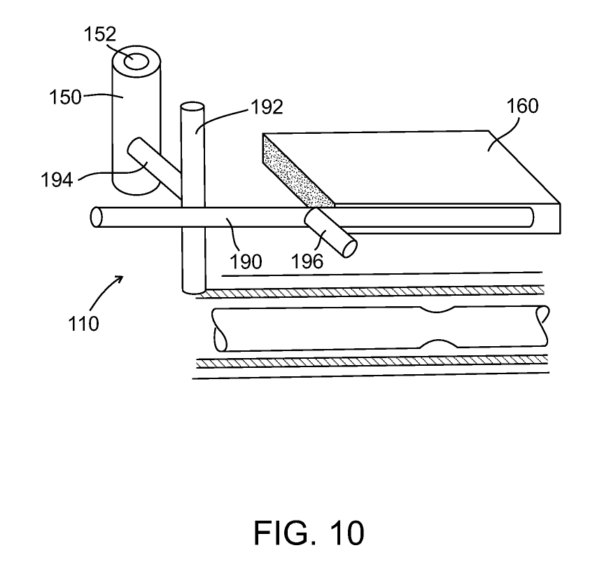

FIG. 10 illustrates another exemplary design of emitter-sensor-assembly 110, incorporating an emitter-sensor element 160 (or a sensor, e.g., for use with an intramedullary light source) and drill sleeve 150 placed in a same horizontal plane so that they cannot both be along the vector through the nail hole, at a same time.

In an exemplary embodiment of the invention, both emitter-sensor element 160 and drill sleeve 150 are connected to a rod 190. Optionally, rod 190 is equipped with a handle 196 used for holding the assembly. Alternatively (not shown), rod 190 may include a coupler for attaching to a tripod or a frame enabling attachment to a steady component (e.g., cart, table, treatment bed).

Arms 192 and 194 connect drill sleeve 150 to rod 190, and may, optionally, provide for relative motion of drill sleeve 150 in the vertical and/or horizontal plane, relative to emitter-sensor element 160. The assembly is connected to an electrical source (not shown) or provides for internal power source (e.g., a battery). During operation emitter-sensor element 160 is placed against the approximate location of the nail hole for which drilling is desired. After sensors of emitter-sensor element 160 detect a hole pattern (e.g., darker area surrounded by illuminated area) of sufficiently equivalent intensity, both emitter-sensor element 160 and drill sleeve 150 are moved a set distance in the plane parallel to the face of emitter-sensor element 160 until the long axis of drill sleeve 150 coincides with the long axis of the nail hole. Optionally, the whole system is first moved so that a known portion of element 160 overlays the nail hole. Movement of parts may be, for example, software controlled and automatic, or by hand. In an exemplary embodiment of the invention, The distance and direction during movement are controlled with some type of location aid unit (e.g., which assists in repositioning an element or in moving an element to the location previously occupied by another element, using, for example, optical encoders, accelerometers and/or position sensors). A signal generator (e.g., visual and/or audible; not shown) may be provided to indicate correct location of the drill sleeve against the nail hole following the said movement. The distance both emitter-sensor element 160 and drill sleeve 150 are moved is based, for example, on the relative distance between them, which is optionally fixed.

A potential advantage of this design (e.g., for a reflective and/or trans-illuminating system) where no aperture is found in the sensor, is that a sensor 160 can provide an electronically scanned map of light output, optionally being used to display an image. In other embodiments, the sensor may be used to collect such information. The collected information may be aligned using a position encoder, so that a map may be built up.

In an exemplary embodiment of the invention, when using emitting component 120 or emitter-sensor element 160 (e.g., intended for placement outside the treated extremity), emitter-sensor-assembly 110 is placed such that a combination of sensor matrix 130 (or sensor matrix 165) and/or emitter 120 (or source-sensor element 160), as available, are located next to the treated extremity, adjacent the approximate area of the holes. Emitter-sensor-assembly 110 is then slightly moved and rotated until the correct location and alignment of nail hole with the line passing through the geometric center of the sensor is achieved, as indicated on the assembly.

Once correct location and alignment are obtained, an incision is made to the skin at the area indicate by the sensor. The bone is exposed, and drill sleeve 150 is optionally advanced towards the bone. Drill sleeve 150 is optionally equipped with sensors and indicators similar to those provided with drill sleeve 80, to provide for fine tuning of the required drilling location and orientation. Once the exact position for drilling is set, drilling may commence via cannulation 152 of the drill sleeve.

In an embodiment of the present invention the targeting system includes circuitry which provides a visual and/or audible alarm to move the emitting component from its location once proper targeting is achieved, and prior to initiation of drilling into the bone. In another embodiment of the present invention said sensors-assembly or emitter-sensor-assembly automatically moves away the emitting component prior to initiation of drilling of the desired hole.

Alternative Exemplary Scanning Based System

FIGS. 11A, 11B and 11C illustrate an alternative design, which optionally combines features from the previously described systems (e.g., systems shown in FIGS. 7, 8 and 10), in accordance with an exemplary embodiment of the invention. This emitter-sensor-assembly 110 provides for light source 120 (or emitter-sensor element 160; not shown) optionally connected to a sensor matrix 165 via a set of rod and arms 140, 142, 144, 146, and optionally equipped with handle 148, as in FIG. 7. As shown, drill sleeve 150 is optionally connected to the system at the same plane as sensor matrix 165 and/or so they interfere in space if moved.

In an exemplary embodiment of the invention, operation is initiated with emitting component 120 (or emitter-sensor element 160) and sensor matrix 165 aligned vertically (FIG. 11A). Once nail hole location and orientation are detected, for example, following a procedure as described above for FIGS. 7 and 8 or FIG. 6, sensor matrix 165, or the combination of sensor matrix 165 and emitting component 120 (or emitter-sensor element 160) are replaced by drill sleeve 150, for example, following the procedure described above for FIG. 10, either automatically, or manually (FIGS. 11B and 11C respectively).

It should be noted that the designs described in FIGS. 6, 7, 8, 9, 10, and 11A-11C provide also for imaging of an anatomy, in accordance with some embodiments of the invention. Instead of nailed bone the target anatomy for imaging is placed against the emitting component and sensor matrix (optionally, the sensor matrix in such case is made of light sensing material (or circuit), such as, but not limited to, CCD or CMOS, or a matrix of diode sensors), and the image is not (only) a hole pattern of sufficiently equivalent intensity, but an image of the illuminated or trans-illuminated anatomy. When used for imaging, the said designs are optionally provided without the optional drill sleeve. They may be provided, optionally, with various surgical tools, connected and moved, in the same manner (for example, but not limited to, a biopsy needle). In the case of imaging, the sensor matrix is optionally connected to a screen, which is optionally formed on a back side of array 120, for example. In the case of imaging an indicator of correct positioning may not be required.

In an exemplary embodiment of the invention, data is collected at multiple relative positions of the sensor and detector and/or at multiple angles relative to the bone. The collected data is processed to generate an image of the anatomy (e.g., hand, wrist or other areas.

In an exemplary embodiment of the invention, when the system is used for anatomy imaging, the source and sensor are placed at different relative angles to each other, either on the same side of the anatomy and/or one opposite sides of the anatomy. When the source and sensor are located on the same side, the source emits radiation towards the anatomy, at different angles relative to the anatomy. The sensor is optionally located at different angles (e.g., not only 0 degrees) relative to the source, for example, at different locations around the source, in a plane parallel to the anatomy. It is expected that the reflections from anatomy be different under different conditions and detectable at said different locations, due to reflection properties of the anatomy. For example, the angles can be, between 1 and 90 degrees, for example, between 10 and 50 degrees, for example, at 2, 4, 6 or more different angular positions and/or at 2, 4, 6 or more different positions around the emitter. Optionally or alternatively, the emitter is moved and the sensor is fixed. In an electronic scanning embodiment, instead of or in addition to motion, data is collected from different detectors and/or using different emitters, as needed.

Optionally, signals are collected and processed in order to build an image which is a collection of illuminated locations in the anatomy from different sides (and/or data collected at different angles. When light is expected to be detected in a certain location (angle) however is detected in a different angle, abnormality is suspected. A table may be used to indicate which angles (e.g., and reflectance levels) are expected for which tissue. The processor may disregard this information, or alternately give a probability weighting to each of the locations in order to decide how to reflect the detected intensity. Optionally or alternatively, a correction for tissue thickness and/or angle of incidence is used.

In a trans-illumination type system (e.g., with a source inside the body or on an opposite side of the anatomy, multiple non-180 degree relative positions may be used. Optionally or alternatively, the anatomy is imaged from multiple sides to provide, for example, two orthogonal planar images and/or a mapping of the surface of a bone.

Optionally, an iris or collimator is used on the detector, and only light that is directed to specific location and/or range of angles is detected.

Optionally, the processing includes de-convoluting the data to provide an image based on an expected spreading of anatomical features due to travel of light through soft tissue.

Exemplary System Mounted on a Power Drill

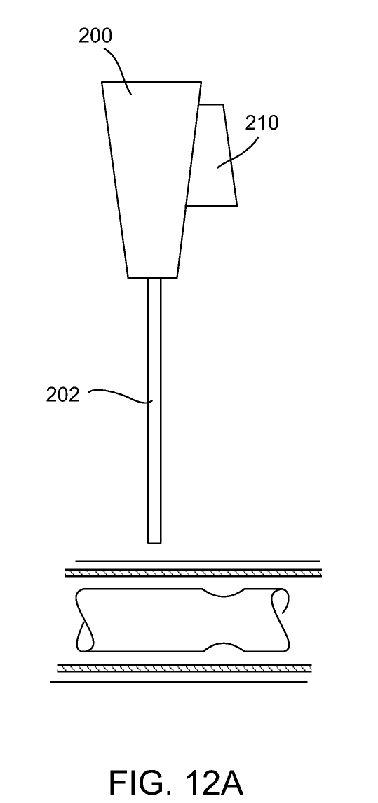

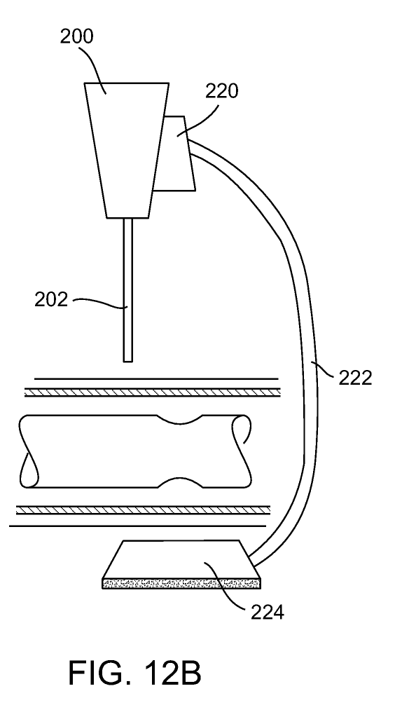

FIGS. 12A and 12B illustrate an emitter-sensor-assembly connected to a power drill 200 (with drill bit 202), in accordance with some exemplary embodiments of the invention. These designs can use the hole locating technologies described above, mounted on a drill (e.g., and weighing less than 1 kg, 500 g, 300 g), rather than using a frame to guide the drill, as described above. The emitter-sensor-assembly is combined of a single unit incorporating both emitting component and sensors (e.g., emitter-sensor element 210, FIG. 12A), or a set of emitting component 220 and sensor matrix 224 connected by arm 222, a gooseneck adjustable arm or other adjustable arm and/or optionally a flexible cable, (FIG. 12B). Again, light source 220 can be replaced by emitter-sensor element 210 in FIG. 12B.

In an exemplary embodiment of the invention, emitter-sensor element 210 (or emitting component 220) is equipped with some type of location aid unit (e.g., to allow it to be repositioned at a pervious location) and a power source. Optionally, this assembly is connected to (e.g., mounted on) the standard power drill used during the operation, and is hand-held. During operation emitter-sensor element 210 (or emitting component 220) and sensor matrix 224, if provided, is placed against the approximate location of the nail hole for which drilling is desired, and is operated, in a similar manner to that described for FIGS. 7, 8, and 10, as applicable. Once all available sensors groups detect hole pattern of sufficiently equivalent intensity (e.g., darker area surrounded by illuminated area, or illuminated area surrounded by darker area, as applicable, e.g., as described above for FIGS. 7, 8, 10) the user moves the power drill to be located against the nail hole location, according to information optionally provided (in visual and/or audible manner) by the emitter-sensor-assembly (based on the location aid unit incorporated into it, combined, optionally with the location of a set reference point in space), and drills the hole using power drill 200 and drill bit 202.

In an exemplary embodiment of the invention, emitter-sensor-assembly 210 or the assembly combined of source 220 (or emitter-sensor element), arm 222, and sensor 224 is connected directly to a power drill and the combination is positioned against the approximate area of the nail hole. The power drill and emitter-sensor-assembly attached to it are then slightly moved and rotated until the correct location and alignment of nail hole with the line passing through the geometric center of the sensor element is achieved, as indicated on the assembly. Once correct location and alignment are obtained, an incision is made to the skin at the area indicate by the sensor. The bone is exposed, and the drill bit connected to the power drill is located against the hole location (e.g., based on information provided by location aid unit, combined, optionally with the location of a set reference point in space). Free-hand drilling is then carried. This procedure can be repeated for additional nail holes.

In an exemplary embodiment of the invention, sensors 210 surround drill 200 and the drill is known to be correctly aligned when they sense light of a uniform and maximal intensity (e.g., an above a threshold), of light transmitted by element 224 through the hole in the nail. Manual manipulation of drill 200 may be used to maintain this situation.

Exemplary Materials

In an embodiment of the present invention all materials incorporated into a tube or enclosure for insertion into the body are biocompatible materials. Optionally, only materials of said housing coming in contact (direct and/or indirect) with the patient and/or physician during operation are made of biocompatible (e.g., “surgical grade”/“implant grade”) materials. In another embodiment of the present invention the materials incorporated into said housing are implant-grade, biodegradable materials (for example, but not limited to, PLA, PLLA).

In an embodiment of the present invention such housing, or, at least, components hereof coming in contact (direct and/or indirect) with the patient and/or physician during operation, comply with at least one method of sterilization (for example, but not limited to, steam sterilization, gamma-radiation sterilization, EtO sterilization).

In an embodiment of the present invention all materials incorporated into components of said sensors-assembly or emitter-sensor-assembly and additional incorporated tools (e.g., drill sleeve) coming in contact (direct and/or indirect) with the patient/physician during operation are biocompatible materials. In an embodiment of the present invention such sensors-assembly or emitter-sensor-assembly, and additional incorporated tools or, at least, components hereof coming in contact (direct and/or indirect) with the patient/physician during operation, comply with at least one method of sterilization (for example, but not limited to, steam sterilization, gamma-radiation sterilization, EtO sterilization).

Exemplary Kits

In an exemplary embodiment of the invention, the targeting system as described herein is multi use. Optionally, a part of the system is provided as a kit for limited time use, optionally with an implant, such as a bone nail and/or locking elements such as bone screws. In one example, the drilling sleeve is disposable. Optionally or alternatively, an insert in the sleeve used for penetrating soft tissue is disposable. Optionally or alternatively, the light source is disposable and may include batteries good for, for example, between 20 and 60 minutes

### Examples

An experiment to determine light transmission was carried out. The medium was bone surrounded by soft tissue. The light source was a laser at NIR (970 nm), with an efficiency of above 50%. Following are some results. It is noted that different results are expected for different limbs, cortical thickness, tissue density, age and/or other physiological properties of the patient and/or extremity treated. The system components were as follows: laser with laser driver, guide wire connected to lens to allow narrow light beam. A power meter was placed on the other side of the medium to collect the energy. For bone, including bone marrow, surrounded by soft tissue and skin with Ø40 mm, the delivered power was 200 mW and the detected power 0.45 μW. for skin, the delivered power was 260 mW and the detected power 38 mW. For, skin with soft tissue with Ø50 mm, the delivered power was 233 mW and the detected power 250 μW.

This shows that physiologically acceptable light levels can be detected outside the body even after they pass through bone and soft tissue.

A detection of the light was performed also with a nail located in the bone, and an in house detector system, which showed values which were more than twice larger in the hole area than along the nail.

General

It is expected that during the life of a patent maturing from this application many relevant bone implants will be developed and the scope of the term “bone implant” (including nail, plate, screw, etc.) is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-40. (canceled)

41. A guiding system adapted to guide drilling through an aperture in an orthopedic implant, comprising:
a frame rigidly interconnecting at least two radiation sources on opposite sides of said aperture and at least two radiation detectors on opposite sides of said aperture, and
circuitry which powers said at least two radiation sources to generate respective radiation beams which are detected by said at least two radiation detectors,
wherein said circuitry is arranged to generate an indication of a change in intensity due to alignment of at least one of said beams with said aperture and at least one of the at least two radiation detectors, and
wherein said circuitry is arranged to generate a signal indicating if a vector pathway for light interconnects said at least one radiation detector with said aperture.

42. The system according to claim 41, wherein said circuitry is further arranged to generate an indication of a correction to apply to improve said alignment.

43. The system according to claim 41, further comprising a drill guide rigidly coupled to said frame and arranged to be movable along said vector pathway.

44. The system according to claim 41, wherein the radiation sources and detectors operate in at least one of the group consisting of: visible, IR, NIR, UV and ionizing radiation wavebands.

45. The system according to claim 41, wherein the change in intensity and the vector pathway are indicated with respect to at least one of the group consisting of: one of the radiation beams passing through human bone surrounding the aperture and overlying soft tissue, and one of the radiation beams reflected from human bone surrounding the aperture through overlying soft tissue.

46. The system according to claim 45, wherein said system is configured to operate with an intramedullary nail for a leg bone and through at least 3 cm of overlaying soft tissue.

47. The system according to claim 45, wherein said system is configured to operate with a bone plate and through at least 1 cm of soft tissue.

48. The system according to claim 45, wherein said circuitry is configured to detect a difference between light passing through at least 0.5 cm of soft tissue and a layer of cortical bone and light passing through a similar thickness of soft tissue and bone, but being blocked by an orthopedic implant.

49. An emitter-sensor assembly comprising at least two emitters and at least two sensors, arranged to be positioned with at least one emitter and at least one sensor on each of at least two sides of an aperture in an implanted orthopedic implant, the emitter-sensor assembly further comprising circuitry arranged to:
power the emitters to emit respective radiation beams,
obtain from the sensors intensity measurements of the emitted radiation,
detect, from the intensity measurements, a change in measured intensity indicative of an alignment of the aperture with at least one of the sensors; and
generate, from the alignment, a signal indicating a vector pathway through the aperture.

50. The emitter-sensor assembly of claim 49, further comprising a frame rigidly and adjustably interconnecting the emitters and the sensors.

51. The emitter-sensor assembly of claim 49, wherein the circuitry is arranged to detect the change with respect to at least one of the group consisting of: one of the radiation beams passing through bone surrounding the aperture and overlying soft tissue, and one of the radiation beams reflected from bone surrounding the aperture through overlying soft tissue.

52. The emitter-sensor assembly of claim 51, further arranged to detect the change when the aperture is within at least one of the group consisting of: a leg bone with at least 3 cm of overlaying soft tissue, a bone plate with at least 1 cm of overlaying soft tissue, and cortical bone with at least 0.5 cm of overlaying soft tissue.

53. A bone drilling system comprising the emitter-sensor assembly of claim 49 and a drill arranged to be guided along the indicated vector pathway.

54. An imaging system comprising the emitter-sensor assembly of claim 49, wherein the circuitry is further arranged to reconstruct, from the intensity measurements, an image of the bone surrounding the aperture.

55. A method comprising:
emitting radiation onto an implanted orthopedic implant and measuring radiation intensity therefrom, wherein the emitting and measuring are each carried out at least two sides of an aperture in the implant;
detecting, from the intensity measurements, a change in the measured intensity indicative of an alignment of the aperture with at least one sensor that measures the radiation, and
generating, from the alignment, a signal indicating a vector pathway through the aperture.

56. The method of claim 55, further comprising moving at least one of an emitting element and a sensing element to yield the change in measured intensity.

57. The method of claim 56, wherein the moving comprises moving rigidly interconnected emitting elements and sensing elements.

58. The method of claim 56, wherein the moving is carried out to change a position of at least one of an emitting element and a sensing element, the position comprising at least one of: an axial location relative to the aperture, a transaxial location relative to the aperture and an angle with respect to an axis going through the aperture.

59. The method of claim 55, further comprising fixating the implant by inserting a screw through the aperture along the indicated vector pathway.

60. The method of claim 55, further comprising reconstructing, from the intensity measurements, an image of bone surrounding the aperture.
